(19) United States
(12) Patent Application Publication
(10) Pub. No.: US 2025/0143548 A1
(43) Pub. Date: May 8, 2025

(54) A DEVICE FOR DETECTING A CONNECTION STATUS AND AN ACTUATOR FOR DETECTING A CONNECTION STATUS

(71) Applicants: Motoki ISHIZAKI, Tokyo (JP); Mitsuharu EZAWA, Tokyo (JP); Yoshimichi IKEDA, Tokyo (JP); Brian NINNI, Irvine, CA (US); CANON KABUSHIKI KAISHA, Tokyo (JP); CANON U.S.A., INC., MELVILLE, NY (US)

(72) Inventors: Motoki Ishizaki, Tokyo (JP); Mitsuharu Ezawa, Kanagawa (JP); Yoshimichi Ikeda, Shizuoka (JP); Brian Ninni, Woburn, MA (US)

(21) Appl. No.: 18/838,478

(22) PCT Filed: Feb. 15, 2023
(86) PCT No.: PCT/US2023/013143

§ 371 Date: Aug. 14, 2024

Related U.S. Application Data
(60) Provisional application No. 63/310,415, filed on Feb. 15, 2022.

(51) Int. Cl. A61B 1/00; A61B 1/005

(52) U.S. Cl. CPC A61B 1/00133; A61B 1/00055; A61B 1/009

(57) ABSTRACT

The device 1 includes a main body drive portion (300) including a plurality of driving sources (M11 to M33), a base unit (200) including a coupling device (21) connected to the main body drive portion, a bendable unit (100) detachably attached to the base unit, and a detection unit configured to detect the coupling portions. In the above configuration, the driving sources are driven, and coupling is detected by sensors.

### CLAIM OF PRIORITY AND INCORPORATION BY REFERENCE

This application claims priority to U.S. Provisional Patent Application No. 63/310,415, which was filed Feb. 15, 2022, and which is incorporated in herein in its entirety.

### TECHNICAL FIELD

The present embodiment relates to a device for detecting a connection status.

### BACKGROUND

PTL 1 describes a medical tool. The medical tool includes an operated portion having a deformable portion and an operating portion that deforms the deformable portion.

In the medical tool, the operated portion and the operating portion are detachably attachable to each other.

### CITATION LIST

### Patent Literature

PTL 1 Japanese Patent Laid-Open No. 2013-248117

In PTL 1, a user couples the operating portion having a power source to a non-operating portion that incorporates a wire configured to move under the power of a power source.

However, when coupling is made, the user is not able to check whether the power of the power source is normally transmitted to the wire. Therefore, the user may use the medical tool while remaining ignorant of the fact that no power is transmitted and may not be able to perform an appropriate operation.

### SUMMARY

One of objects is to provide a detection unit that detects whether power transmission coupling is made at the time when the operating portion and the non-operating portion are shifted into a coupled state, and a device including the detection unit.

One of the embodiments related to the present application is a device comprising: a base including a driving source and a coupling portion; a bendable body detachably attached to the base, the bendable body including a bendable portion configured to bend and a drive wire allowed to be coupled to the coupling portion and configured to bend the bendable portion; a force sensor provided in the base and that is connected to the coupling portion, the force sensor being connected to the drive wire while the drive wire is coupled to the coupling portion; and a detector configured to detect a state of coupling of the coupling portion with the drive wire, wherein while the base is coupled with the coupling portion, the detector detects the state of coupling using an output value of the force sensor while the drive wire is driven by the driving source.

As described above, according to the embodiments, it is possible to provide a detection unit that, in a case where the coupling portion and the drive wire are coupled to each other, detects whether the driving source and the coupling portion are coupled to each other and driving force is transmitted to the drive wire, and a medical device including the detection unit.

### BRIEF DESCRIPTION OF DRAWINGS

FIG. 1 is an overall view of a medical system.

FIG. 2 is a perspective view that shows a medical device and a support.

FIG. 3 is a view that illustrates a catheter.

FIG. 4 is a view that illustrates a catheter unit.

FIG. 5 is a view that illustrates a base unit and a wire drive portion.

FIG. 6 is a view that illustrates the wire drive portion, a coupling device, and a bend drive portion.

FIG. 7 is a view that illustrates attachment of the catheter unit.

FIG. 8 is a view that illustrates coupling of the catheter unit with the base unit.

FIG. 9 is an exploded view that illustrates coupling of the catheter unit with the base unit.

FIG. 10 is a view that illustrates fixing of a drive wire by a coupling portion.

FIG. 11 is a view that illustrates fixing of the drive wire with the coupling portion.

FIG. 12 is a view that illustrates fixing of the drive wire with the coupling portion.

FIG. 13 is a view that illustrates fixing of the drive wire with the coupling portion.

FIG. 14 is a view that illustrates fixing of the drive wire with the coupling portion.

FIG. 15 is a view that illustrates the catheter unit and the base unit.

FIG. 16 is a view that illustrates operations of an operating portion.

FIG. 17 is a sectional view that illustrates operations of the operating portion.

FIG. 18 is a view that illustrates a strain body of a detection unit.

FIG. 19 is a view that illustrates sticking of strain gauges to the strain body.

FIG. 20 is a view that illustrates the strain body and substrate of the detection unit.

FIG. 21 is a flowchart of a series of detection steps by the detection unit.

FIG. 22 is a flowchart of detection steps by strain gauges.

FIG. 23 is a block diagram of detection of coupling by current sensors.

FIG. 24 is a flowchart of detection of coupling by the current sensors.

FIG. 25 is a view that shows a schematic configuration example of a continuum robot according to a third embodiment of the present invention.

FIG. 26 is a view that shows a schematic configuration example of a control system of the continuum robot according to the third embodiment of the present invention.

FIG. 27 is a graph that shows the slidability of the continuum robot according to the present invention.

### DESCRIPTION OF EMBODIMENTS

Hereinafter, the configuration of the present invention will be illustrated with reference to the drawings. The dimensions, materials, and shapes of components that will be described in the present embodiments, the arrangement of the components, and the like, should be changed as needed depending on the configuration of an apparatus to which the present invention is applied, various conditions, or the like.

### First Embodiment

### Medical System and Medical Device

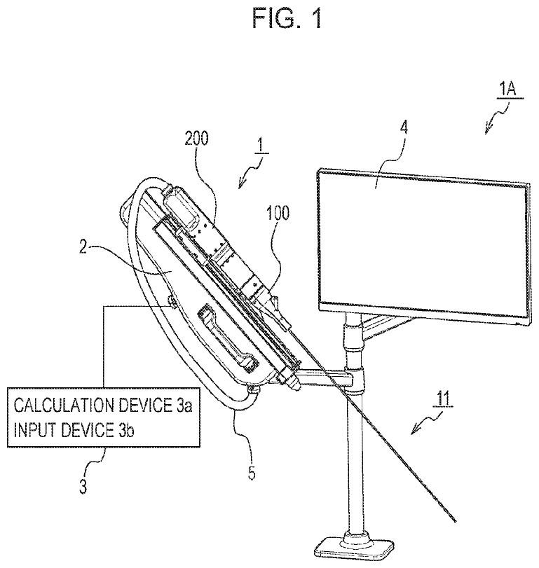

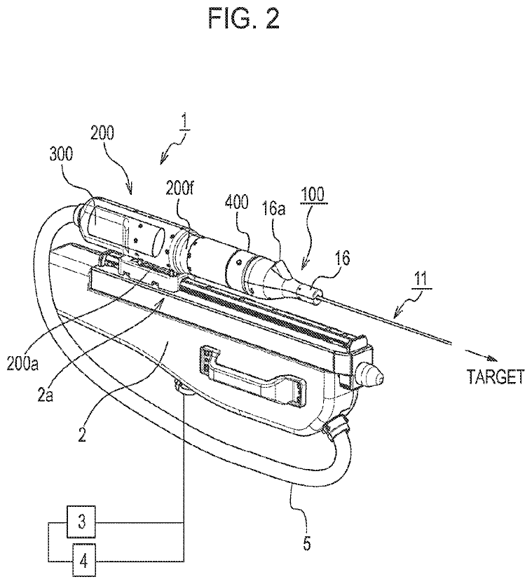

A medical system 1A and a medical device 1 will be described with reference to FIGS. 1 and 2. FIG. 1 is an overall view of the medical system 1A. FIG. 2 is a perspective view that shows the medical device 1 and a support 2.

The medical system 1A includes the medical device 1, the support 2 to which the medical device 1 is attached, and a controller 3 that controls the medical device 1. In the present embodiment, the medical system 1A includes a monitor 4 serving as a display apparatus.

The medical device 1 includes a catheter unit (bendable unit) 100 including a catheter 11 serving as a bendable element, and a base unit (a drive unit or an attached unit) 200. The catheter unit 100 is configured to be detachably attachable to the base unit 200.

In the present embodiment, a user of the medical system 1A and the medical device 1 is able to do some work, such as observing the inside of a target, collecting various samples from the inside of the target, and treating the inside of the target, by inserting the catheter 11 into the inside of the target. As one of embodiments, a user is able to insert the catheter 11 into the inside of a patient as a target. Specifically, by inserting the catheter 11 into a bronchus via the oral cavity or nasal cavity of a patient, it is possible to do some work, such as observation, collection, and removal of a lung tissue.

The catheter 11 can be used as a guide (sheath) that guides a medical tool for doing the above work. Examples of the medical tool (tool) include an endoscope, a forceps, and an ablation device. The catheter 11 itself may have the functions of the above-described medical tools.

In the present embodiment, the controller 3 includes a calculation device 3a and an input device 3b. The input device 3b receives a command or input for operating the catheter 11. The calculation device 3a includes a storage that stores a program and various data for controlling a catheter, a random access memory, and a central processing unit for running the program. The controller 3 may include an output unit that outputs a signal for displaying an image on the monitor 4.

As shown in FIG. 2, in the present embodiment, the medical device 1 is electrically connected to the controller 3 via the support 2 and a cable 5 that couples the base unit 200 of the medical device 1 with the support 2. The medical device 1 and the controller 3 may be directly connected by a cable. The medical device 1 and the controller 3 may be wirelessly connected to each other.

The medical device 1 is detachably attached to the support 2 via the base unit 200. More specifically, in the medical device 1, an attachment portion (connecting portion) 200a of the base unit 200 is detachably attached to a movable stage (receiving portion) 2a of the support 2. Even in a state where the attachment portion 200a of the medical device 1 is detached from the movable stage 2a, connection of the medical device 1 with the controller 3 is maintained such that the medical device 1 is controllable by the controller 3. In the present embodiment, even in a state where the attachment portion 200a of the medical device 1 is detached from the movable stage 2a, the medical device 1 and the support 2 are connected by the cable 5.

A user is able to manually move the medical device 1 in a state where the medical device 1 is detached from the support 2 (a state where the medical device 1 is detached from the movable stage 2a) and insert the catheter 11 into the inside of a target.

A user is able to use the medical device 1 in a state where the catheter 11 is inserted in a target and the medical device 1 is attached to the support 2.

Specifically, when the movable stage 2a moves in a state where the medical device 1 is attached to the movable stage 2a, the medical device 1 moves. Then, an operation to move the catheter 11 in a direction to be inserted into the target and an operation to move the catheter 11 in a direction to be pulled out from the target are performed. Movement of the movable stage 2a is controlled by the controller 3.

The attachment portion 200a of the base unit 200 includes an unlock switch (not shown) and a detachment switch (not shown). In a state where the attachment portion 200a is attached to the movable stage 2a, a user is able to manually move the medical device 1 along a guide direction of the movable stage 2a while holding down the unlock switch. In other words, the movable stage 2a includes a guide configuration to guide movement of the medical device 1. When the user stops pressing the unlock switch, the medical device 1 is fixed to the movable stage 2a. On the other hand, when the detachment switch is pressed in a state where the attachment portion 200a is attached to the movable stage 2a, a user is able to detach the medical device 1 from the movable stage 2a.

A single switch may have the function of the unlock switch and the function of the detachment switch. When the unlock switch is provided with a mechanism of switching between a pressed down state and a non-pressed down state, a user does not need to hold down the unlock switch when manually sliding the medical device 1.

In a state where the attachment portion 200a is attached to the movable stage 2a and the unlock switch or the detachment switch is not pressed, the medical device 1 is fixed to the movable stage 2a and is moved by the movable stage 2a driven by a motor (not shown).

The medical device 1 includes a wire drive portion (a linear member drive portion, a line drive portion, or a main body drive portion) 300 for driving the catheter 11. In the present embodiment, the medical device 1 is a robot catheter device that drives the catheter 11 with the wire drive portion 300 controlled by the controller 3.

The controller 3 can control the wire drive portion 300 and perform an operation to bend the catheter 11. In the present embodiment, the wire drive portion 300 is incorporated in the base unit 200. More specifically, the base unit 200 includes a base housing 200f that accommodates the wire drive portion 300. In other words, the base unit 200 includes the wire drive portion 300. The wire drive portion 300 and the base unit 200 may be collectively referred to as a catheter drive apparatus (a base apparatus or a main body).

In an extending direction of the catheter 11, an end portion at which the distal end of the catheter 11 to be inserted into a target is disposed is referred to as a distal end. In the extending direction of the catheter 11, an opposite side to the distal end is referred to as a proximal end.

The catheter unit 100 has a proximal end cover 16 that covers the proximal end side of the catheter 11. The proximal end cover 16 has a tool hole 16a. A medical tool is allowed to be inserted into the catheter 11 via the tool hole 16a.

As described above, in the present embodiment, the catheter 11 has the function of a guide device for guiding a medical tool to a desired position inside a target.

For example, in a state where an endoscope is inserted in the catheter 11, the catheter 11 is inserted to an intended position inside a target. At this time, at least one of manual operation of a user, movement of the movable stage 2a, and drive of the catheter 11 with the wire drive portion 300 is used. After the catheter 11 reaches the intended position, the endoscope is pulled out from the catheter 11 via the tool hole 16a. Then, a medical tool is inserted through the tool hole 16a, and some work, such as collecting various samples from the inside of the target and treating the inside of the target, is performed.

As will be described later, the catheter unit 100 is detachably attached to the catheter drive apparatus (a base apparatus or a main body), more specifically, the base unit 200. After the medical device 1 is used, a user is able to detach the catheter unit 100 from the base unit 200, attach a new catheter unit 100 to the base unit 200, and use the medical device 1 again.

As shown in FIG. 2, the medical device 1 includes an operating portion 400. In the present embodiment, the operating portion 400 is provided in the catheter unit 100. The operating portion 400 is operated by a user when the catheter unit 100 is fixed to the base unit 200 or when the catheter unit 100 is detached from the base unit 200.

By connecting the endoscope inserted in the catheter 11 with the monitor 4, the monitor 4 can display an image taken by the endoscope on the monitor 4. By connecting the monitor 4 with the controller 3, the status of the medical device 1 and information related to control over the medical device 1 can be displayed on the monitor 4. For example, the position of the catheter 11 inside a target or information related to navigation for the catheter 11 inside a target can be displayed on the monitor 4. The monitor 4 and both the controller 3 and the endoscope may be connected by wire or may be connected by wireless. The monitor 4 and the controller 3 may be directly connected via the support 2.

### Catheter

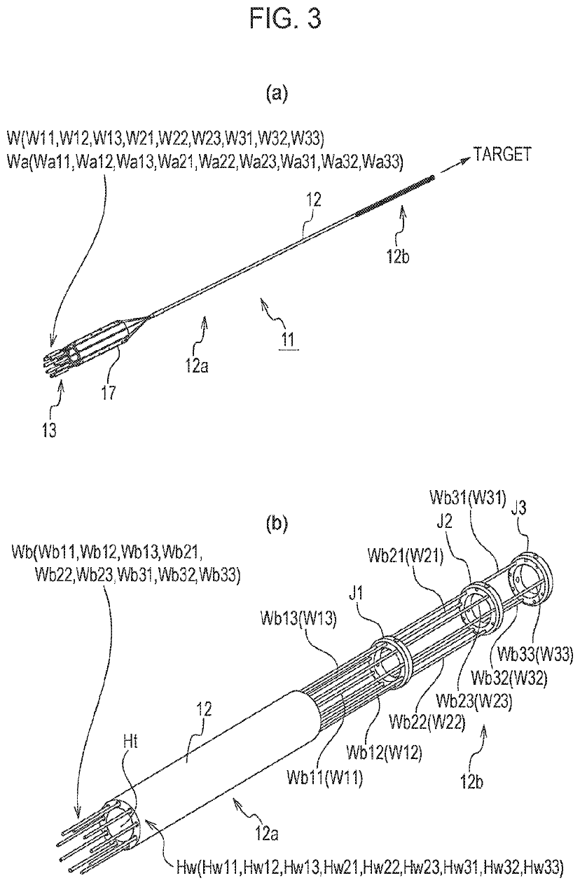

The catheter 11 serving as a bendable body will be described with reference to FIG. 3. FIG. 3 is a view that illustrates the catheter 11. FIG. 3(a) is a view that illustrates the whole of the catheter 11. FIG. 3(b) is an enlarged view of the catheter 11.

The catheter 11 includes a bendable portion (a bendable body or a catheter main body) 12 and a bend drive portion (catheter drive portion) 13 configured to bend the bendable portion 12. The bend drive portion 13 is configured to bend the bendable portion 12 upon receiving the driving force of the wire drive portion 300 via a coupling device 21 (described later).

The catheter 11 extends along the direction in which the catheter 11 is inserted into a target. The extending direction (longitudinal direction) of the catheter 11 is the same as an extending direction (longitudinal direction) of the bendable portion 12 and an extending direction (longitudinal direction) of each of first to ninth drive wires (W11 to W33) (described later).

The bend drive portion 13 includes the plurality of drive wires (drive lines, linear members, or linear actuators) connected to the bendable portion 12.

Specifically, the bend drive portion 13 includes the first drive wire W11, the second drive wire W12, the third drive wire W13, the fourth drive wire W21, the fifth drive wire W22, the sixth drive wire W23, the seventh drive wire W31, the eighth drive wire W32, and the ninth drive wire W33.

Each of the first to ninth drive wires (W11 to W33) includes a held portion (a held shaft or a rod) Wa. Specifically, the first drive wire W11 includes the first held portion Wa11. The second drive wire W12 includes the second held portion Wa12. The third drive wire W13 includes the third held portion Wa13. The fourth drive wire W21 includes the fourth held portion Wa21. The fifth drive wire W22 includes the fifth held portion Wa22. The sixth drive wire W23 includes the sixth held portion Wa23. The seventh drive wire W31 includes the seventh held portion Wa31. The eighth drive wire W32 includes the eighth held portion Wa32. The ninth drive wire W33 includes the ninth held portion Wa33.

In the present embodiment, each of the first to ninth held portions (Wa11 to Wa33) has the same shape.

Each of the first to ninth drive wires (W11 to W33) includes a flexible wire body (a line body or a linear body) Wb. Specifically, the first drive wire W11 includes the first wire body Wb11. The second drive wire W12 includes the second wire body Wb12. The third drive wire W13 includes the third wire body Wb13. The fourth drive wire W21 includes the fourth wire body Wb21. The fifth drive wire W22 includes the fifth wire body Wb22. The sixth drive wire W23 includes the sixth wire body Wb23. The seventh drive wire W31 includes the seventh wire body Wb31. The eighth drive wire W32 includes the eighth wire body Wb32. The ninth drive wire W33 includes the ninth wire body Wb33.

In the present embodiment, each of the first to third wire bodies (Wb11 to Wb13) has the same shape. Each of the fourth to sixth wire bodies (Wb21 to Wb23) has the same shape. Each of the seventh to ninth wire bodies (Wb31 to Wb33) has the same shape. In the present embodiment, the first to ninth wire bodies (Wb11 to Wb33) have the same shape.

Each of the first to ninth held portions (Wa11 to Wa33) is fixed to a corresponding one of the first to ninth wire bodies (Wb11 to Wb33) at the proximal end of the corresponding one of the first to ninth wire bodies (Wb11 to Wb33).

The first to ninth drive wires (W11 to W33) are inserted into the bendable portion 12 via a wire guide 17 and fixed.

In the present embodiment, the material of each of the first to ninth drive wires (W11 to W33) is a metal. The material of each of the first to ninth drive wires (W11 to W33) may be a resin. The material of each of the first to ninth drive wires (W11 to W33) may include metal and resin.

Of the first to ninth drive wires (W11 to W33), a selected one may be referred to as a drive wire W. In the present embodiment, each of the first to ninth drive wires (W11 to W33) has the same shape except for the length of each of the first to ninth drive wires (Wb11 to Wb33).

In the present embodiment, the bendable portion 12 has flexibility and is a tubular member having a passage Ht for inserting a medical tool.

A wall of the bendable portion 12 includes a plurality of wire holes for respectively passing the first to ninth drive wires (W11 to W33). Specifically, the wall of the bendable portion 12 includes the first wire hole Hw11, the second wire hole Hw12, the third wire hole Hw13, the fourth wire hole Hw21, the fifth wire hole Hw22, the sixth wire hole Hw23, the seventh wire hole Hw31, the eighth wire hole Hw32, and the ninth wire hole Hw33. The first to ninth wire holes Hw (Hw11 to Hw33) are respectively in correspondence with the first to ninth drive wires (W11 to W33). The numeral suffixed to the sign Hw represents the numeral of a corresponding one of the drive wires. For example, the first drive wire W11 is inserted into the first wire hole Hw11.

Of the first to ninth wire holes (Hw11 to Hw33), a selected one may be referred to as a wire hole Hw. In the present embodiment, each of the first to ninth wire holes (Hw11 to Hw33) has the same shape.

The bendable portion 12 has an intermediate region 12a and a bendable region 12b. The bendable region 12b is disposed at the distal end of the bendable portion 12. A first guide ring J1, a second guide ring J2, and a third guide ring J3 are disposed in the bendable region 12b. The bendable region 12b means a region capable of controlling the degree and direction of bending of the bendable portion 12 by moving the first guide ring J1, the second guide ring J2, and the third guide ring J3 with the bend drive portion 13. FIG. 3(b) is a view drawn by omitting part of the bendable portion 12 covering the first to third guide rings (J1 to J3).

In the present embodiment, the bendable portion 12 includes a plurality of sub-rings (not shown). In the bendable region 12b, the first guide ring J1, the second guide ring J2, and the third guide ring J3 are fixed to the wall of the bendable portion 12. In the present embodiment, the sub-rings are respectively disposed on the proximal side with respect to the first guide ring J1, between the first guide ring J1 and the second guide ring J2, and between the second guide ring J2 and the third guide ring J3.

A medical tool is guided to the distal end of the catheter 11 by the passage Ht, the first to third guide rings (J1 to J3), and the plurality of sub-rings.

Each of the first to ninth drive wires (W11 to W33) is fixed to a corresponding one of the first to third guide rings (J1 to J3) through the intermediate region 12a.

Specifically, the first drive wire W11, the second drive wire W12, and the third drive wire W13 extend through the plurality of sub-rings and are fixed to the first guide ring J1. The fourth drive wire W21, the fifth drive wire W22, and the sixth drive wire W23 extend through the first guide ring J1 and the plurality of sub-rings and are fixed to the second guide ring J2. The seventh drive wire W31, the eighth drive wire W32, and the ninth drive wire W33 extend through the first guide ring J1, the second guide ring J2, and the plurality of sub-rings and are fixed to the third guide ring J3.

The medical device 1 is capable of bending the bendable portion 12 in a direction that intersects with the extending direction of the catheter 11 by driving the bend drive portion 13 with the wire drive portion 300. Specifically, by moving each of the first to ninth drive wires (W11 to W33) in the extending direction of the bendable portion 12, the bendable region 12b of the bendable portion 12 is bent in a direction that intersects with the extending direction via the first to third guide rings (J1 to J3).

A user is able to insert the catheter 11 to an intended part inside a target by using at least any one of moving the medical device 1 manually or with the movable stage 2a and bending the bendable portion 12.

In the present embodiment, the bendable portion 12 is bent by moving the first to third guide rings (J1 to J3) with the first to ninth drive wires (W11 to W33); however, the present invention is not limited to this configuration. Any one or two of the first to third guide rings (J1 to J3) and the drive wires fixed to them may be omitted.

For example, the catheter 11 may have such a configuration that the first to sixth drive wires (W11 to W23) and the first and second guide rings (J1, J2) are omitted and only the seventh to ninth drive wires (W31 to W33) and the third guide ring J3 are provided.

Alternatively, the catheter 11 may have such a configuration that the first to third drive wires (W11 to W13) and the first guide ring J1 are omitted and only the fourth to ninth drive wires (W21 to W33) and the second and third guide rings (J2, J3) are provided.

Alternatively, the catheter 11 may have such a configuration that a single guide ring is driven by two drive wires. In this case as well, the number of guide rings may be one or may be more than one.

### Catheter Unit

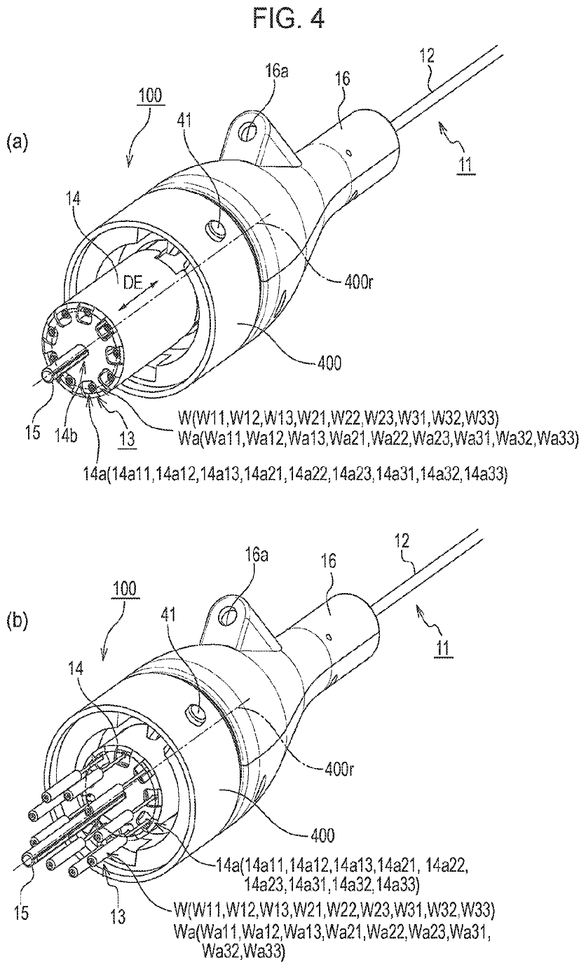

The catheter unit 100 will be described with reference to FIG. 4.

FIG. 4 is a view that illustrates the catheter unit 100. FIG. 4(a) is a view that illustrates the catheter unit 100 in a state where a wire cover 14 (described later) is at a cover position. FIG. 4(b) is a view that illustrates the catheter unit 100 in a state where the wire cover 14 (described later) is at a retracted position.

The catheter unit 100 includes the catheter 11 including the bendable portion 12 and the bend drive portion 13, and a proximal end cover 16 that supports the proximal end of the catheter 11. The catheter unit 100 includes the cover (wire cover) 14 for covering and protecting the first to ninth drive wires (W11 to W33) serving as the plurality of drive wires.

The catheter unit 100 is detachably attachable to the base unit 200 along an attaching and detaching direction DE. The direction in which the catheter unit 100 is attached to the base unit 200 and the direction in which the catheter unit 100 is detached from the base unit 200 are parallel to the attaching and detaching direction DE.

The proximal end cover (a frame, a bendable portion housing, or a catheter housing) 16 is a cover covering part of the catheter 11. The proximal end cover 16 has the tool hole 16a for inserting a medical tool into the passage Ht of the bendable portion 12.

The wire cover 14 has a plurality of wire cover holes (cover holes) for respectively passing the first to ninth drive wires (W11 to W33). The wire cover 14 has the first wire cover hole 14a11, the second wire cover hole 14a12, the third wire cover hole 14a13, the fourth wire cover hole 14a21, the fifth wire cover hole 14a22, the sixth wire cover hole 14a23, the seventh wire cover hole 14a31, the eighth wire cover hole 14a32, and the ninth wire cover hole 14a33. The first to ninth wire cover holes (14a11 to 14a33) are respectively in correspondence with the first to ninth drive wires (W11 to W33). The numeral suffixed to the sign 14a represents the numeral of a corresponding one of the drive wires. For example, the first drive wire W11 is inserted into the first wire cover hole 14a11.

Of the first to ninth wire cover holes (14a11 to 14a33), a selected one may be referred to as a wire cover hole 14a. In the present embodiment, each of the first to ninth wire cover holes (14a11 to 14a33) has the same shape.

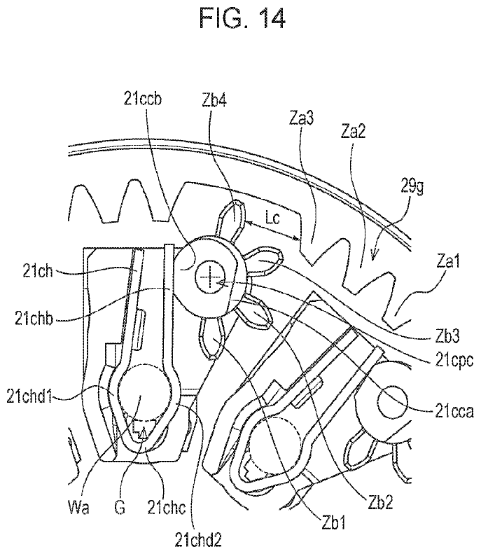

The wire cover 14 can be moved to a cover position (see FIG. 14(a)) where the first to ninth drive wires (W11 to W33) are covered and a retracted position (see FIG. 14(b)) retracted from the cover position. The retracted position may also be referred to as an exposed position where the first to ninth drive wires (W11 to W33) are exposed.

Before the catheter unit 100 is attached to the base unit 200, the wire cover 14 is located at the cover position. When the catheter unit 100 is attached to the base unit 200, the wire cover 14 moves from the cover position to the retracted position along the attaching and detaching direction DE.

In the present embodiment, after the wire cover 14 is moved from the cover position to the retracted position, the wire cover 14 is retained at the retracted position.

Therefore, even when the catheter unit 100 is attached to the base unit 200 and then the catheter unit 100 is detached from the base unit 200, the wire cover 14 is retained at the retracted position.

However, after the wire cover 14 is moved from the cover position to the retracted position, the wire cover 14 may be configured to return to the cover position. For example, the catheter unit 100 may include an urging member that urges the wire cover 14 from the retracted position toward the cover position. In this case, when the catheter unit 100 is detached from the base unit 200 after the catheter unit 100 is attached to the base unit 200, the wire cover 14 is moved from the retracted position to the cover position.

When the wire cover 14 is at the retracted position, the first to ninth held portions (Wa11 to Wa33) of the first to ninth drive wires (W11 to W33) protrude with respect to the wire cover 14. As a result, coupling of the bend drive portion 13 with the coupling device 21 (described later) is allowed. When the wire cover 14 is at the retracted position, the first to ninth held portions (Wa11 to Wa33) of the first to ninth drive wires (W11 to W33) protrude from the first to ninth wire cover holes (14a11 to 14a33). More specifically, the first to ninth held portions (Wa11 to Wa33) protrude from the first to ninth wire cover holes (14a11 to 14a33) in an attachment direction Da (described later).

As shown in FIG. 4(b), the first to ninth drive wires (W11 to W33) are arranged along a circle (imaginary circle) having a predetermined radius.

In the present embodiment, the catheter unit 100 includes a key shaft (a key or a catheter-side key) 15. In the present embodiment, the key shaft 15 extends in the attaching and detaching direction DE. The wire cover 14 has a shaft hole 14b through which the key shaft 15 extends. The key shaft 15 is engageable with a key receiving portion 22 (described later). When the key shaft 15 is engaged with the key receiving portion 22, movement of the catheter unit 100 with respect to the base unit 200 is limited within a predetermined range in the circumferential direction of the circle (imaginary circle) along which the first to ninth drive wires (W11 to W33) are arranged.

In the present embodiment, when viewed in the attaching and detaching direction DE, the first to ninth drive wires (W11 to W33) are disposed outside the key shaft 15 so as to surround the key shaft 15. In other words, the key shaft 15 is disposed inside the circle (imaginary circle) along which the first to ninth drive wires (W11 to W33) are arranged. Therefore, the key shaft 15 and the first to ninth drive wires (W11 to W33) can be disposed in a space-saving manner.

In the present embodiment, the catheter unit 100 includes the operating portion 400. The operating portion 400 is configured to be movable (rotatable) with respect to the proximal end cover 16 and the bend drive portion 13. The operating portion 400 is rotatable around a rotation axis 400r. The rotation axis 400r of the operating portion 400 extends in the attaching and detaching direction DE.

In a state where the catheter unit 100 is attached to the base unit 200, the operating portion 400 is configured to be movable (rotatable) with respect to the base unit 200. More specifically, the operating portion 400 is configured to be movable (rotatable) with respect to the base housing 200f, the wire drive portion 300, and the coupling device 21 (described later).

### Base Unit

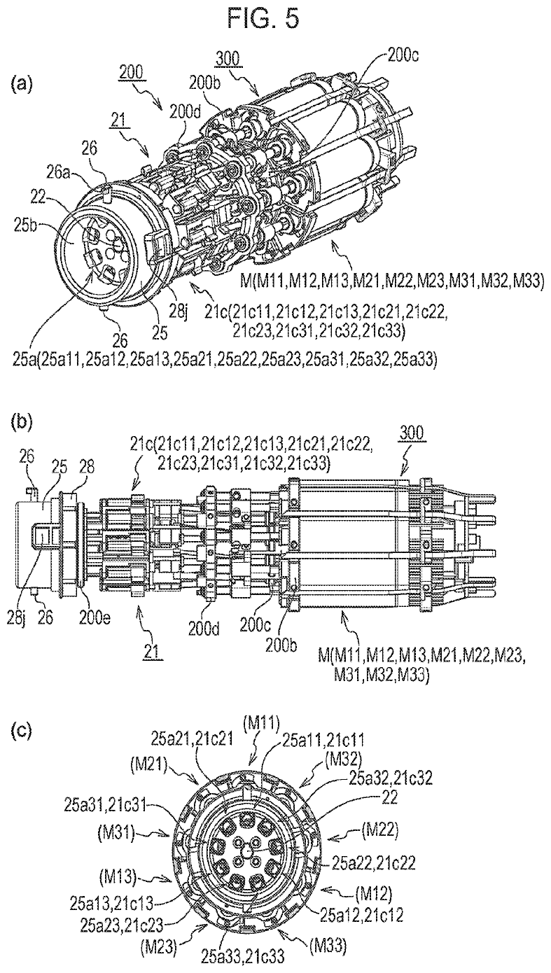

The base unit 200 and the wire drive portion 300 will be described with reference to FIG. 5.

FIG. 5 is a view that illustrates the base unit 200 and the wire drive portion 300. FIG. 5(a) is a perspective view that shows the internal structure of the base unit 200. FIG. 5(b) is a side view that shows the internal structure of the base unit 200. FIG. 5(c) is a view of the base unit 200 when viewed along the attaching and detaching direction DE.

As described above, the medical device 1 includes the base unit 200 and the wire drive portion 300. In the present embodiment, the wire drive portion 300 is accommodated in the base housing 200f and is provided inside the base unit 200. In other words, the base unit 200 includes the wire drive portion 300.

The wire drive portion 300 includes a plurality of driving sources (motors). In the present embodiment, the wire drive portion 300 includes the first driving source M11, the second driving source M12, the third driving source M13, the fourth driving source M21, the fifth driving source M22, the sixth driving source M23, the seventh driving source M31, the eighth driving source M32, and the ninth driving source M33.

Of the first to ninth driving sources (M11 to M33), a selected one may be referred to as a driving source M. In the present embodiment, each of the first to ninth driving sources (M11 to M33) has the same configuration.

The base unit 200 includes the coupling device 21. The coupling device 21 is accommodated in the base housing 200f. The coupling device 21 is connected to the wire drive portion 300. The coupling device 21 includes a plurality of coupling portions. In the present embodiment, the coupling device 21 includes the first coupling portion 21c11, the second coupling portion 21c12, the third coupling portion 21c13, the fourth coupling portion 21c21, the fifth coupling portion 21c22, the sixth coupling portion 21c23, the seventh coupling portion 21c31, the eighth coupling portion 21c32, and the ninth coupling portion 21c33.

Of the first to ninth coupling portions (21c11 to 21c33), a selected one may be referred to as a coupling portion 21c. In the present embodiment, each of the first to ninth coupling portions (21c11 to 21c33) has the same configuration.

Each of the plurality of coupling portions is connected to a corresponding one of the plurality of driving sources and is driven by the corresponding one of the plurality of driving sources. Specifically, the first coupling portion 21c11 is connected to the first driving source M11 and is driven by the first driving source M11. The second coupling portion 21c12 is connected to the second driving source M12 and is driven by the second driving source M12. The third coupling portion 21c13 is connected to the third driving source M13 and is driven by the third driving source M13. The fourth coupling portion 21c21 is connected to the fourth driving source M21 and is driven by the fourth driving source M21. The fifth coupling portion 21c22 is connected to the fifth driving source M22 and is driven by the fifth driving source M22. The sixth coupling portion 21c23 is connected to the sixth driving source M23 and is driven by the sixth driving source M23. The seventh coupling portion 21c31 is connected to the seventh driving source M31 and is driven by the seventh driving source M31. The eighth coupling portion 21c32 is connected to the eighth driving source M32 and is driven by the eighth driving source M32. The ninth coupling portion 21c33 is connected to the ninth driving source M33 and is driven by the ninth driving source M33.

As will be described later, the bend drive portion 13 including the first to ninth drive wires (W11 to W33) is coupled to the coupling device 21. The bend drive portion 13 receives the driving force of the wire drive portion 300 via the coupling device 21 to bend the bend drive portion 12.

The drive wire W is coupled to the coupling portion 21c via the held portion Wa. Each of the plurality of drive wires is coupled to a corresponding one of the plurality of coupling portions.

Specifically, the first held portion Wa11 of the first drive wire W11 is coupled to the first coupling portion 21c11. The second held portion Wa12 of the second drive wire W12 is coupled to the second coupling portion 21c12. The third held portion Wa13 of the third drive wire W13 is coupled to the third coupling portion 21c13. The fourth held portion Wa21 of the fourth drive wire W21 is coupled to the fourth coupling portion 21c21. The fifth held portion Wa22 of the fifth drive wire W22 is coupled to the fifth coupling portion 21c22. The sixth held portion Wa23 of the sixth drive wire W23 is coupled to the sixth coupling portion 21c23. The seventh held portion Wa31 of the seventh drive wire W31 is coupled to the seventh coupling portion 21c31. The eighth held portion Wa32 of the eighth drive wire W32 is coupled to the eighth coupling portion 21c32. The ninth held portion Wa33 of the ninth drive wire W33 is coupled to the ninth coupling portion 21c33.

The base unit 200 includes a base frame 25. The base frame 25 has a plurality of insertion holes for respectively passing the first to ninth drive wires (W11 to W33). The base frame 25 has the first insertion hole 25a11, the second insertion hole 25a12, the third insertion hole 25a13, the fourth insertion hole 25a21, the fifth insertion hole 25a22, the sixth insertion hole 25a23, the seventh insertion hole 25a31, the eighth insertion hole 25a32, and the ninth insertion hole 25a33. The first to ninth insertion holes (25a11 to 25a33) are respectively in correspondence with the first to ninth drive wires (W11 to W33). The numeral suffixed to the sign 25a represents the numeral of a corresponding one of the drive wires. For example, the first drive wire W11 is inserted into the first insertion hole 25a11.

Of the first to ninth insertion holes (25a11 to 25a33), a selected one may be referred to as an insertion hole 25a. In the present embodiment, each of the first to ninth insertion holes (25a11 to 25a33) has the same shape.

The base frame 25 has an attachment opening 25b into which the wire cover 14 is inserted. The first to ninth insertion holes (25a11 to 25a33) are disposed at the bottom portion of the attachment opening 25b.

In addition, the base unit 200 includes a motor frame 200b, a first bearing frame 200c, a second bearing frame 200d, and a third bearing frame 200e. The motor frame 200b, the first bearing frame 200c, the second bearing frame 200d, and the third bearing frame 200e are coupled to one another.

The base frame 25 has a key receiving portion (a key hole, a base-side key, or a main body-side key) 22 for receiving the key shaft 15. When the key shaft 15 and the key receiving portion 22 are engaged with each other, the catheter unit 100 is attached to the base unit 200 in proper phase.

When the key shaft 15 is engaged with the key receiving portion 22, movement of the catheter unit 100 with respect to the base unit 200 is limited within a predetermined range in the circumferential direction of the circle (imaginary circle) along which the first to ninth drive wires (W11 to W33) are arranged.

As a result, each of the first to ninth drive wires (W11 to W33) is engaged with a corresponding one of the first to ninth insertion holes (25a11 to 25a33) and a corresponding one of the first to ninth coupling portions (21c11 to 21c33). In other words, engagement of the drive wire W with a non-corresponding one of the insertion holes 25a and a non-corresponding one of the coupling portions 21c is prevented.

A user is able to properly couple each of the first to ninth drive wires (W11 to W33) with a corresponding one of the first to ninth coupling portions (21c11 to 21c33) by engaging the key shaft 15 with the key receiving portion 22. Therefore, a user is able to easily attach the catheter unit 100 to the base unit 200.

In the present embodiment, the key shaft 15 has a protruding portion that protrudes in a direction to intersect with the attaching and detaching direction DE, and the key receiving portion 22 has a recess portion into which the protruding portion is inserted. A position in which the protruding portion and the recess portion are engaged with each other in the circumferential direction is a position in which each of the drive wires W is engaged with a corresponding one of the insertion holes 25a and a corresponding one of the coupling portions 21c.

The key shaft 15 may be disposed in any one of the base unit 200 and the catheter unit 100, and the key receiving portion 22 may be disposed in the other. For example, the key shaft 15 may be disposed at the base unit 200 side, and the key receiving portion 22 may be disposed at the catheter unit 100 side.

The base unit 200 has a joint 28 that includes a joint engagement portion 28j. The base frame 25 includes a lock shaft 26 having a lock protrusion 26a. The functions of them will be described later.

Coupling of Motor with Drive Wire

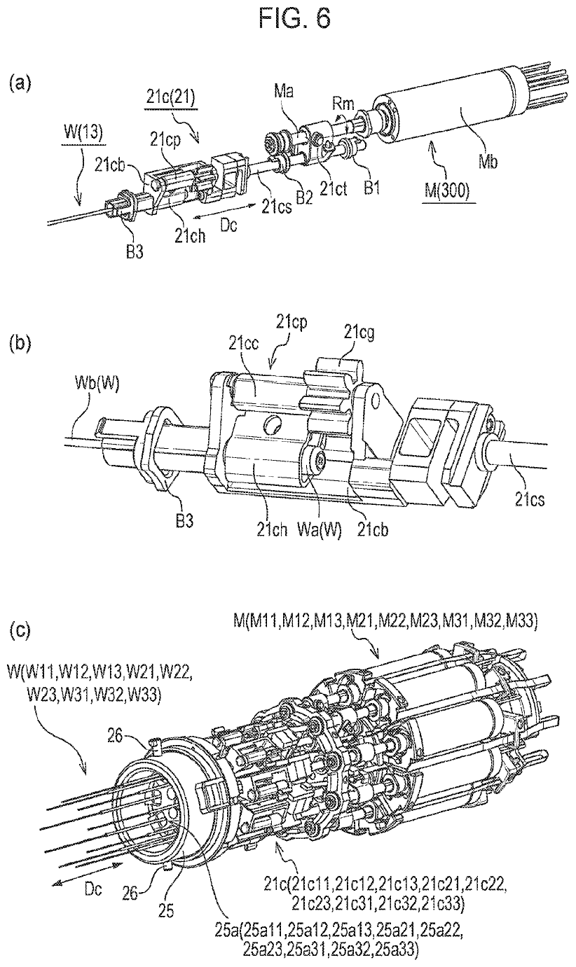

Coupling among the wire drive portion 300, the coupling device 21, and the bend drive portion 13 will be described with reference to FIG. 6.

FIG. 6 is a view that illustrates the wire drive portion 300, the coupling device 21, and the bend drive portion 13. FIG. 6(a) is a perspective view of the driving source M, the coupling portion 21c, and the drive wire W. FIG. 6(b) is an enlarged view of the coupling portion 21c and the drive wire W. FIG. 6(c) is a perspective view that shows coupling among the wire drive portion 300, the coupling device 21, and the bend drive portion 13.

In the present embodiment, the configuration in which each of the first to ninth drive wires (W11 to W33) is coupled to a corresponding one of the first to ninth coupling portions (21c11 to 21c33) is the same. The configuration in which each of the first to ninth coupling portions (21c11 to 21c33) is connected to a corresponding one of the first to ninth driving sources (M11 to M33) is the same. Therefore, in the following description, the configuration in which the drive wire W, the coupling portion 21c, and the driving source M are connected will be described by using the one drive wire W, the one coupling portion 21c, and the one driving source M.

As shown in FIG. 6(a), the driving source M includes an output shaft Ma and a motor main body Mb that rotates the output shaft Ma in a rotation direction Rm. A spiral groove is provided on the surface of the output shaft Ma. The output shaft Ma has a so-called screw shape. The motor main body Mb is fixed to the motor frame 200b.

The coupling portion 21c has a tractor 21ct connected to the output shaft Ma and a tractor support shaft 21cs that supports the tractor 21ct. The tractor support shaft 21cs is connected to a coupling base 21cb.

The coupling portion 21c includes a leaf spring 21ch serving as a holding portion for holding the held portion Wa of the drive wire W. The drive wire W is engaged with the coupling portion 21c through the insertion hole 25a. More specifically, the held portion Wa is engaged with the leaf spring 21ch. As will be described later, the leaf spring 21ch can be placed in a state where the held portion Wa is clamped and fixed (fixed state) and a state where the held portion Wa is released (released state).

The coupling portion 21c includes a pressing member 21cp. The pressing member 21cp has a gear portion 21cg meshed with an internal gear 29 (described later) and a cam 21cc serving as a pressing portion for pressing the leaf spring 21ch.

As will be described later, the cam 21cc can move with respect to the leaf spring 21ch. When the cam 21cc moves, the leaf spring 21ch is switched between the fixed state and the released state.

The coupling portion 21c is supported by a first bearing B1, a second bearing B2, and a third bearing B3. The first bearing B1 is supported by the first bearing frame 200c of the base unit 200. The second bearing B2 is supported by the second bearing frame 200d of the base unit 200. The third bearing B3 is supported by the third bearing frame 200e of the base unit 200. Therefore, when the motor shaft Ma rotates in the rotation direction Rm, rotation of the coupling portion 21c around the motor shaft Ma is restricted. The first bearing B1, the second bearing B2, and the third bearing B3 are provided for each of the first to ninth coupling portions (21c11 to 21c33).

Since rotation of the coupling portion 21c around the motor shaft Ma is restricted, when the motor shaft Ma rotates, a force along the rotational axis direction of the motor shaft Ma is applied to the tractor 21ct by the spiral groove of the motor shaft Ma. As a result, the coupling portion 21c moves along the rotational axis direction of the motor shaft Ma (Dc direction). When the coupling portion 21c moves, the drive wire W moves, and the bendable portion 12 bends.

In other words, the motor shaft Ma and the tractor 21ct make up a so-called feed screw that converts rotational motion transmitted from the driving source M to linear motion by a screw. In the present embodiment, the motor shaft Ma and the tractor 21ct are slide screws.

Alternatively, the motor shaft Ma and the tractor 21ct may be ball screws.

As shown in FIG. 6(c), by attaching the catheter unit 100 to the base unit 200, each of the first to ninth drive wires (W11 to W33) and a corresponding one of the first to ninth coupling portions (21c11 to 21c33) are coupled to each other.

The controller 3 is capable of controlling the first to ninth driving sources (M11 to M33) independently of each other. In other words, a selected one driving source of the first to ninth driving sources (M11 to M33) is allowed to independently operate or stop regardless of whether the other driving sources are stopped. In other words, the controller 3 is capable of controlling each of the first to ninth drive wires (W11 to W33) independently of one another. As a result, each of the first to third guide rings (J1 to J3) is controlled independently of one another, and the bendable region 12b of the bendable portion 12 is allowed to bend in a selected direction.

### Attachment of Catheter Unit

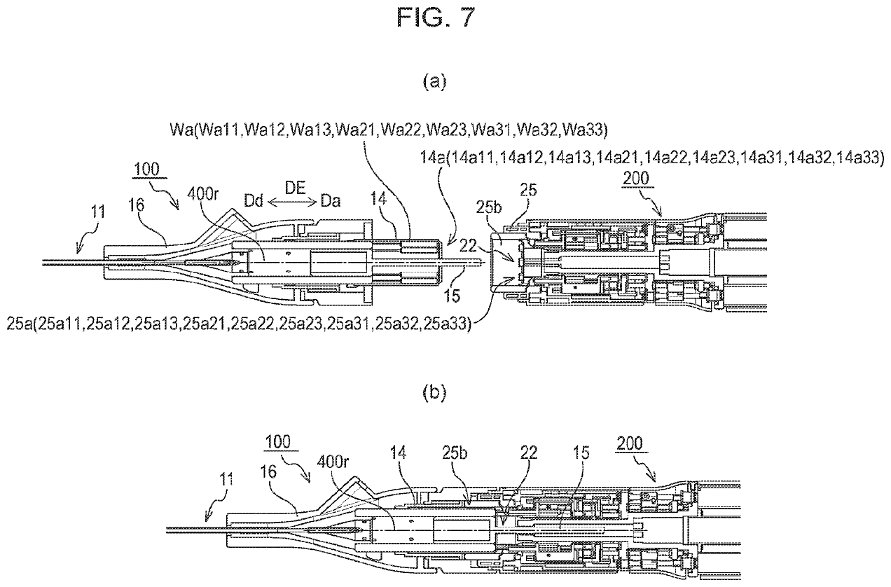

An operation to attach the catheter unit 100 to the base unit 200 will be described with reference to FIG. 7.

FIG. 7 is a view that illustrates attachment of the catheter unit 100. FIG. 7(a) is a view before the catheter unit 100 is attached to the base unit 200. FIG. 7(b) is a view after the catheter unit 100 is attached to the base unit 200.

In the present embodiment, the attaching and detaching direction DE of the catheter unit 100 is the same as the direction of the rotation axis 400r of the operating portion 400. In the attaching and detaching direction DE, the direction in which the catheter unit 100 is attached to the base unit 200 is referred to as the attachment direction Da. In the attaching and detaching direction DE, the direction in which the catheter unit 100 is detached from the base unit 200 (a direction opposite to the attachment direction Da) is referred to as a detachment direction Dd.

As shown in FIG. 7(a), in a state before the catheter unit 100 is attached to the base unit 200, the wire cover 14 is placed at the cover position. At this time, the wire cover 14 covers the first to ninth drive wires (W11 to W33) such that the first to ninth held portions (Wa11 to Wa33) do not protrude from the first to ninth wire cover holes (14a11 to 14a33) of the wire cover 14. Therefore, in a state before the catheter unit 100 is attached to the base unit 200, the first to ninth drive wires (W11 to W33) are protected.

When the catheter unit 100 is attached to the base unit 200, the key shaft 15 is engaged with the key receiving portion 22. The key shaft 15 protrudes from the wire cover 14. In the present embodiment, in a state where the key shaft 15 has reached the entrance of the key receiving portion 22, the wire cover 14 is not engaged with the attachment opening 25b. In other words, when the phase of the catheter unit 100 with respect to the base unit 200 is a phase in which the key shaft 15 and the key receiving portion 22 are not engaged with each other, the wire cover 14 is not engaged with the attachment opening 25b, and the state where the wire cover 14 is placed at the cover position is maintained. Therefore, even when the catheter unit 100 is moved such that the key shaft 15 and the key receiving portion 22 are engaged with each other, the first to ninth drive wires (W11 to W33) are protected.

When the key shaft 15 and the key receiving portion 22 are engaged with each other and the catheter unit 100 is moved in the attachment direction Da with respect to the base unit 200, the catheter unit 100 is attached to the base unit 200. When the catheter unit 100 is attached to the base unit 200, the wire cover 14 is moved to the retracted position. In the present embodiment, the wire cover 14 contacts with the base frame 25 to move from the cover position to the retracted position (see FIG. 7(b)).

More specifically, when the catheter unit 100 is attached, the wire cover 14 contacts with the base frame 25 to stop. In this state, by moving the catheter unit 100 in the attachment direction Da, the wire cover 14 relatively moves with respect to a part other than the wire cover 14 in the catheter unit 100. As a result, the wire cover 14 moves from the cover position to the retracted position.

The wire cover 14 moves from the cover position to the retracted position, while the held portion Wa of the drive wire W protrudes from the wire cover hole 14a of the wire cover 14 and is inserted into the insertion hole 25a. Then, the held portion Wa is engaged with the leaf spring 21ch of the coupling portion 21c (see FIG. 6(b)).

In a state where the catheter unit 100 is just attached to the base unit 200, the catheter unit 100 can be detached by moving the catheter unit 100 in the detachment direction Dd with respect to the base unit 200. As will be described later, in a state where the catheter unit 100 is just attached to the base unit 200, fixing of the drive wire W with the coupling portion 21c is in an unlocked state.

In a state where the catheter unit 100 is attached to the base unit 200, detachment of the catheter unit 100 from the base unit 200 by operating the operating portion 400 is prevented. In addition, by operating the operating portion 400 in a state where the catheter unit 100 is attached to the base unit 200, the bend drive portion 13 is fixed to the coupling device 21, and the bend drive portion 13 is coupled to the wire drive portion 300 via the coupling device 21.

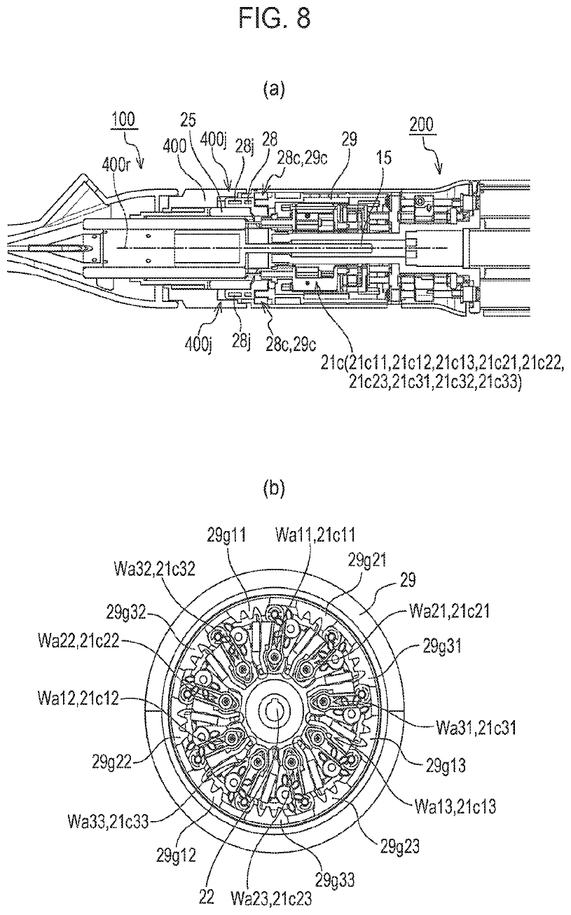

Fixing of Bend Drive Portion and Unlocking of Fixing A configuration for fixing the bend drive portion 13 to the coupling device 21 and a configuration for unlocking fixing of the bend drive portion 13 with the coupling device 21 will be described with reference to FIGS. 8, 9, 10, 11, 12, 13, and 14.

FIG. 8 is a view that illustrates coupling of the catheter unit 100 with the base unit 200. FIG. 8(a) is a sectional view of the catheter unit 100 and the base unit 200. FIG. 8(a) is a sectional view of the catheter unit 100 and the base unit 200, taken along the rotation axis 400r. FIG. 8(b) is a sectional view of the base unit 200. FIG. 8(b) is a sectional view of the base unit 200, taken in a direction orthogonal to the rotation axis 400r at a part of the coupling portion 21c.

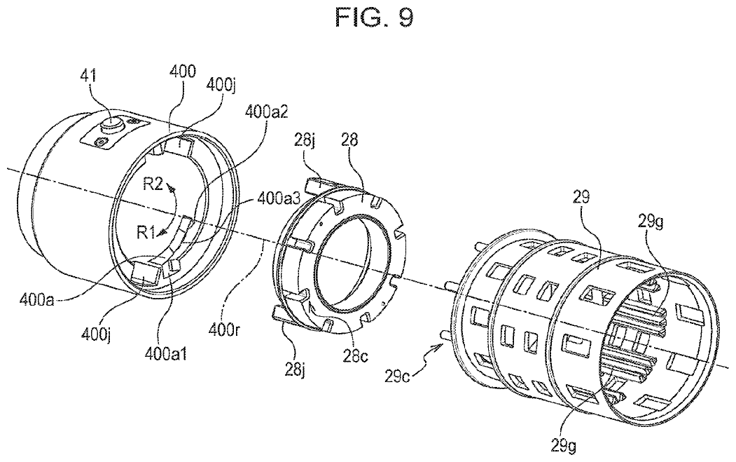

FIG. 9 is an exploded view that illustrates coupling of the catheter unit 100 with the base unit 200.

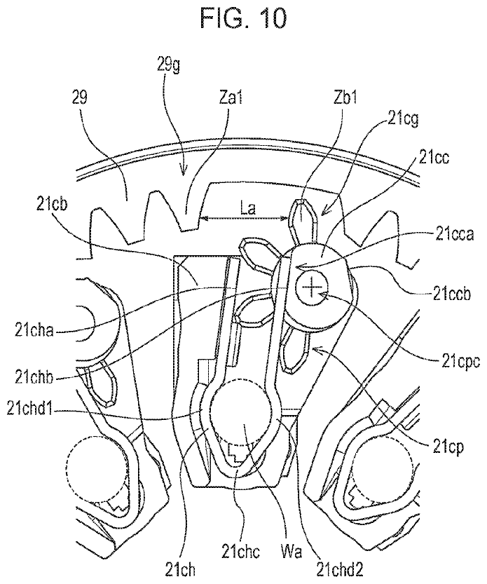

FIGS. 10, 11, 12, 13, and 14 are views that illustrate fixing of the drive wire W with the coupling portion 21c.

As shown in FIGS. 8(a) and 9, the base unit 200 includes the joint (an intermediate member or a second transmission member) 28, and the internal gear 29 serving as a movable gear (an interlocking gear, a transmission member, or a first transmission member) that is in interlocking wit the operating portion 400 via the joint 28.

The joint 28 has a plurality of transmitting portions 28c. The internal gear 29 has a plurality of transmitted portions 29c. The plurality of transmitting portions 28c is engaged with the plurality of transmitted portions 29c, and, when the joint 28 rotates, rotation of the joint 28 is transmitted to the internal gear 29.

When the catheter unit 100 is attached to the base unit 200, the engagement portion 400j provided in the operating portion 400 is engaged with the joint engagement portion 28j of the joint 28. When the operating portion 400 rotates, rotation of the operating portion 400 is transmitted to the joint 28. The operating portion 400, the joint 28, and the internal gear 29 rotate in the same direction.

The internal gear 29 has a plurality of tooth portions for switching between a state where each of the first to ninth coupling portions (21c11 to 21c33) fixes a corresponding one of the first to ninth drive wires (W11 to W33) and a state where each of the first to ninth coupling portions (21c11 to 21c33) releases a corresponding one of the first to ninth drive wires (W11 to W33). Each of the plurality of tooth portions (a working portion or a gear switching portion) of the internal gear 29 is engaged with the gear portion 21cg of the pressing member 21cp of each of the first to ninth coupling portions (21c11 to 21c33).

Specifically, in the present embodiment, the internal gear 29 has the first tooth portion 29g11, the second tooth portion 29g12, the third tooth portion 29g13, the fourth tooth portion 29g21, the fifth tooth portion 29g22, the sixth tooth portion 29g23, the seventh tooth portion 29g31, the eighth tooth portion 29g32, and the ninth tooth portion 29g33. The first to ninth tooth portions (29g11 to 29g33) are formed with a gap from each other.

The first tooth portion 29g11 meshes with the gear portion 21cg of the first coupling portion 21c11. The second tooth portion 29g12 meshes with the gear portion 21cg of the second coupling portion 21c12. The third tooth portion 29g13 meshes with the gear portion 21cg of the third coupling portion 21c13. The fourth tooth portion 29g21 meshes with the gear portion 21cg of the fourth coupling portion 21c21. The fifth tooth portion 29g22 meshes with the gear portion 21cg of the fifth coupling portion 21c22. The sixth tooth portion 29g23 meshes with the gear portion 21cg of the sixth coupling portion 21c23. The seventh tooth portion 29g31 meshes with the gear portion 21cg of the seventh coupling portion 21c31. The eighth tooth portion 29g32 meshes with the gear portion 21cg of the eighth coupling portion 21c32. The ninth tooth portion 29g33 meshes with the gear portion 21cg of the ninth coupling portion 21c33.

Of the first to ninth tooth portions (29g11 to 29g33), a selected one may be referred to as a tooth portion 29g. In the present embodiment, each of the first to ninth tooth portions (29g11 to 29g33) has the same configuration.

In the present embodiment, the configuration in which each of the first to ninth drive wires (W11 to W33) is coupled to a corresponding one of the first to ninth coupling portions (21c11 to 21c33) is the same. The configuration in which each of the first to ninth coupling portions (21c11 to 21c33) is connected to a corresponding one of the first to ninth tooth portions (29g11 to 29g33) is the same. Therefore, in the following description, the configuration in which the drive wire W, the coupling portion 21c, and the tooth portion 29g are connected will be described by using the one drive wire W, the one coupling portion 21c, and the one tooth portion 29g.

In each of the first to ninth coupling portions (21c11 to 21c33), when the gear portion 21cg is moved by the internal gear 29, the pressing member 21cp rotates, and the cam 21cc moves to a pressing position or to a retracted position retracted from the pressing position.

By rotating the operating portion 400, the internal gear 29 rotates. When the internal gear 29 rotates, each of the first to ninth coupling portions (21c11 to 21c33) operates. In other words, with an operation to rotate the one operating portion 400, the first to ninth coupling portions (21c11 to 21c33) are actuated.

The operating portion 400 is allowed to move to a fixed position (lock position) and a detachment position in a state where the catheter unit 100 is attached to the base unit 200. As will be described later, the operating portion 400 is allowed to move to the unlock position in a state where the catheter unit 100 is attached to the base unit 200. In the rotation direction of the operating portion 400, the unlock position is located between the fixed position and the detachment position. In a state where the operating portion 400 is placed at the detachment position, the catheter unit 100 is attached to the base unit 200.

In a state where the catheter unit 100 is attached to the base unit 200, the drive wire W is not fixed (locked) to the coupling portion 21c. This state is referred to as an unlocked state of the coupling portion 21c. A state where the drive wire W is fixed (locked) to the coupling portion 21c is referred to as a locked state of the coupling portion 21c.

An operation to fix the drive wire W to the coupling portion 21c will be described with reference to FIGS. 10, 11, 12, 13, and 14.

In a state after the catheter unit 100 is attached to the base unit 200 and before the operating portion 400 is operated, the catheter unit 100 is allowed to be detached from the base unit 200. Hereinafter, a state where the catheter unit 100 is allowed to be detached from the base unit 200 is referred to as a detachable state.

FIG. 10 is a view that shows a state of the internal gear 29 and the coupling portion 21c in the detachable state. FIG. 10 is a view that shows the internal gear 29 and the coupling portion 21c in a state where the operating portion 400 is placed at the fixed position.

The leaf spring 21ch of the coupling portion 21c has a fixed portion 21cha fixed to the coupling base 21cb, and a pressed portion 21chb that contacts with the cam 21cc of the pressing member 21cp. The leaf spring 21ch has a first part 21chd1 and a second part 21chd2. When the catheter unit 100 is attached to the base unit 200, the held portion Wa is inserted between the first part 21chd1 and the second part 21chd2.

The cam 21cc has a holding surface 21cca and a pressing surface 21ccb. In a radial direction of rotation of the pressing member 21cp, the holding surface 21cca is disposed at a position closer to a rotation center 21cpc of the pressing member 21cp than the pressing surface 21ccb.

As shown in FIG. 10, in the detachable state (a state where the operating portion 400 is at the detachment position), the leaf spring 21ch is held at a position at which the pressed portion 21chb is in contact with the holding surface 21cca. A tooth Za1 of the internal gear 29 and a tooth Zb1 of the gear portion 21cg are stopped in a state where there is a clearance La therebetween.

In the rotation direction of the operating portion 400, a direction in which the operating portion 400 heads from the detachment position for the unlock position and the fixed position is referred to as a lock direction (fixing direction), and a direction in which the operating portion 400 heads from the fixed position for the unlock position and the detachment position is referred to as an unlock direction. The operating portion 400 rotates in the unlock direction from the unlock position and moves to the detachment position. The operating portion 400 rotates in the lock direction from the unlock position to move to the fixed position.

In a state where the catheter unit 100 is attached to the base unit 200 and the operating portion 400 is at the detachment position, the coupling portion 21c is in an unlocked state, and fixing of the drive wire W with the coupling portion 21c is unlocked.

When the coupling portion 21c is in the unlocked state, the cam 21cc is placed at the retracted position retracted from the pressing position (described later). At this time, fixing of the held portion Wa with the leaf spring 21ch is unlocked. A force that the first part 21chd1 and the second part 21chd2 fasten the held portion Wa when the coupling portion 21c is in the unlocked state is less than a force that the first part 21chd1 and the second part 21chd2 fasten the held portion Wa when the coupling portion 21c is in the locked state.

When the catheter unit is moved in the detachment direction Dd with respect to the base unit 200 while the coupling portion 21c is in the unlocked state, the held portion Wa can be pulled out from between the first part 21chd1 and the second part 21chd2.

It is desirable that no force that the first part 21chd1 and the second part 21chd2 fasten the held portion Wa be generated (a state where the magnitude is zero) when the coupling portion 21c is in the unlocked state. It is desirable that a clearance be formed between the held portion Wa and at least any one of the first part 21chd1 and the second part 21chd2 when the coupling portion 21c is in the unlocked state.

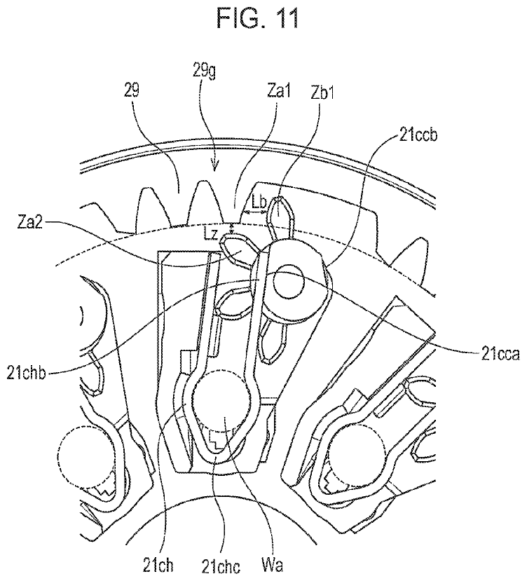

FIG. 11 is a view that shows a state of the internal gear 29 and the coupling portion 21c when the operating portion 400 is rotated in the lock direction from the detachment position. FIG. 11 is a view that shows a state of the internal gear 29 and the coupling portion 21c in a state where the operating portion 400 is at the unlock position.

When the operating portion 400 is rotated in the lock direction in a state where the operating portion 400 is at the detachment position (FIG. 10), the internal gear 29 rotates in the clockwise direction. Then, the operating portion 400 is placed at the unlock position.

Even when the operating portion 400 is rotated, the key shaft 15 and the key receiving portion 22 are engaged with each other, so rotation of the whole (except the operating portion 400) of the catheter unit 100 with respect to the base unit 200 is restricted. In other words, the operating portion 400 is rotatable with respect to the whole (except the operating portion 400) of the catheter unit 100 and the base unit 200 in a state where the whole (except the operating portion 400) of the catheter unit 100 and the base unit 200 are stopped.

When the internal gear 29 rotates in the clockwise direction, the clearance between the tooth Za1 of the internal gear 29 and the tooth Zb1 of the gear portion 21cg reduces from a clearance La to a clearance Lb.

A tooth Zb2 of the gear portion 21cg is disposed at a position spaced a clearance Lz apart from a tip circle (dashed line) of the tooth portion 29g of the internal gear 29. Therefore, the internal gear 29 is rotatable without interference with the tooth Zb2. On the other hand, the coupling portion 21c is maintained in the same state (unlocked state) as the state shown in FIG. 10.

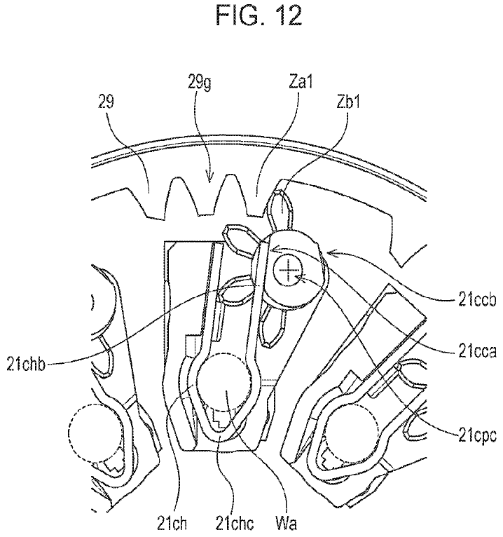

When the operating portion 400 is further rotated from the state shown in FIG. 11 in the lock direction, the internal gear 29 is further rotated in the clockwise direction. FIG. 12 shows a state of the internal gear 29 and the coupling portion 21c at that time.

FIG. 12 is a view that shows a state of the internal gear 29 and the coupling portion 21c when the operating portion 400 is rotated in the lock direction from the unlock position.

As shown in FIG. 12, when the operating portion 400 is rotated in the lock direction from the unlock position, the tooth Za1 of the internal gear 29 and the tooth Zb1 of the gear portion 21cg contact with each other. On the other hand, the coupling portion 21c is maintained in the unlocked state that is the same state as the state shown in FIGS. 10 and 11.

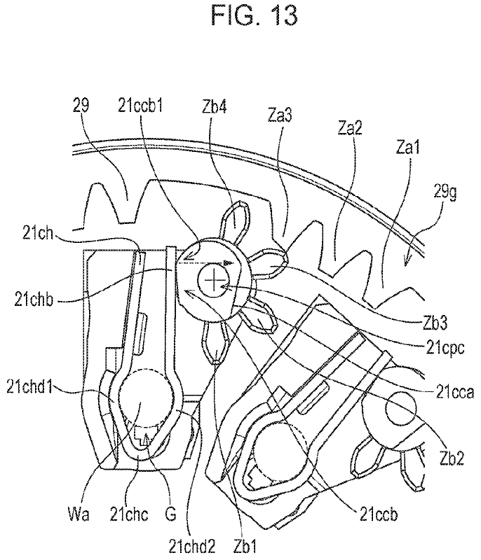

FIG. 13 is a view that shows a state where the pressing member 21cp is rotated as a result of rotation of the operating portion 400 in the lock direction.

As shown in FIG. 13, when the operating portion 400 is further rotated from the state shown in FIG. 12 in the lock direction, the internal gear 29 is further rotated in the clockwise direction.

The internal gear 29 shifts from the state of FIG. 12 to the state of FIG. 13, with the result that the internal gear 29 rotates the gear portion 21cg in the clockwise direction. When the gear portion 21cg rotates, the holding surface 21cca separates from the pressed portion 21chb, and the pressing surface 21ccb approaches the pressed portion 21chb. Then, the first part 21chd1 and the second part 21chd2 begin to clamp the held portion Wa.

Then, while the pressed portion 21chb is pressed by a corner 21ccb1 disposed at the end portion of the pressing surface 21ccb, a tooth Za3 of the internal gear 29 moves to a position separated from a tooth Zb3 of the gear portion 21cg. At this time, the held portion Wa is clamped by the first part 21chd1 and the second part 21chd2.

When the tooth Za3 of the internal gear 29 separates from the tooth Zb3 of the gear portion 21cg, transmission of driving force from the internal gear 29 to the gear portion 21cg stops. At this time, the corner 21ccb1 of the cam 21cc receives a reaction force from the leaf spring 21ch.

In the radial direction of rotation of the pressing member 21cp, the reaction force of the leaf spring 21ch, applied to the corner 21ccb1, is applied at a position spaced apart from the rotation center 21cpc of the pressing member 21cp, and the pressing member 21cp rotates in the clockwise direction. At this time, the pressing member 21cp rotates in the same direction as the direction to be rotated by the internal gear 29 that rotates in the clockwise direction.

FIG. 14 is a view that shows a state of the internal gear 29 and the coupling portion 21c in a state where the operating portion 400 is at the fixed position.

As shown in FIG. 14, the pressing member 21cp further rotates from the state shown in FIG. 13 upon receiving the reaction force of the leaf spring 21ch.

As shown in FIG. 14, the pressing member 21cp stops in a state where the pressing surface 21ccb of the cam 21cc and the pressed portion 21chb of the leaf spring 21ch are in area contact with each other. In other words, the pressing surface 21ccb and the surface of the pressed portion 21chb are in a state of being arranged in the same plane.

At this time, the coupling portion 21c is in a locked state. When the coupling portion 21c is in the locked state, the cam 21cc of the pressing member 21cp is placed at the pressing position, and the pressing surface 21ccb presses the pressed portion 21chb.

When the coupling portion 21c is in the locked state, the held portion Wa is clamped by the first part 21chd1 and the second part 21chd2. In other words, the leaf spring 21ch is pressed by the cam 21cc, and the held portion Wa is fastened by the leaf spring 21ch. As a result, the held portion Wa is fixed by the leaf spring 21ch.

In the present embodiment, in the leaf spring 21ch, the first part 21chd1 and the second part 21chd2 press the held portion Wa at positions spaced apart from each other. In addition, a bent portion 21chc is disposed between the first part 21chd1 and the second part 21chd2 to connect the first part 21chd1 and the second part 21chd2. The bent portion 21chc is disposed with a gap G from the held portion Wa. Thus, the held portion Wa is stably fixed by the first part 21chd1 and the second part 21chd2.

The material of the leaf spring 21ch may be a resin or a metal and is preferably a metal.

When the coupling portion 21c is in the locked state, the held portion Wa is restricted from being pulled out from between the first part 21chd1 and the second part 21chd2.

The tooth Za3 of the internal gear 29 and a tooth Zb4 of the gear portion 21cg are stopped at positions where there is a clearance Lc therebetween.

When fixing of the drive wire W to the coupling portion 21c is unlocked, the operating portion 400 placed at the fixed position is rotated in the unlock direction. At this time, the internal gear 29 rotates from the state shown in FIG. 14 in the counterclockwise direction. When the internal gear 29 rotates in the counterclockwise direction, the tooth Za3 of the internal gear 29 contacts with the tooth Zb4 of the gear portion 21cg, and the pressing member 21cp is rotated in the counterclockwise direction.

By further rotating the internal gear 29 in the counterclockwise direction, fixing of the drive wire W with the coupling portion 21c is unlocked. The operations of the internal gear 29 and the pressing member 21cp at this time are operations reverse to the above-described operations. In other words, fixing of the drive wire W with the coupling portion 21c is unlocked by the operation reverse to the operation at the time of fixing the above-described drive wire W with the coupling portion 21c.

The above-described operations are performed in each of the first to ninth coupling portions (21c11 to 21c33). In other words, in the course in which the operating portion 400 moves from the detachment position to the fixed position, the first to ninth coupling portions (21c11 to 21c33) shift from the unlocked state to the locked state by movement (rotation) of the operating portion 400. In the course in which the operating portion 400 moves from the fixed position to the detachment position, the first to ninth coupling portions (21c11 to 21c33) shift from the locked state to the unlocked state by movement (rotation) of the operating portion 400. In other words, a user is able to switch the plurality of coupling portions between the unlocked state and the locked state by operating the single operating portion 400.

In other words, it is not necessary that each of the plurality of coupling portions includes an operating portion for switching between the unlocked state and the locked state and a user operates the plurality of coupling portions. Therefore, a user is able to easily attach and detach the catheter unit 100 to the base unit 200. In addition, the medical device 1 is simplified.

A state where each of the first to ninth drive wires (W11 to W33) is fixed by a corresponding one of the first to ninth coupling portions (21c11 to 21c33) is referred to as a first state. A state where fixing of each of the first to ninth drive wires (W11 to W33) with a corresponding one of the first to ninth coupling portions (21c11 to 21c33) is unlocked is referred to as a second state.

Interlocking with the movement of the operating portion 400, the state is switched between the first state and the second state. In other words, interlocking with the movement of the operating portion 400 between the detachment position and the fixed position, the state is switched between the first state and the second state.

The internal gear 29 is configured to interlock with the operating portion 400. In the present embodiment, the joint 28 functions as a transmission member for interlocking the operating portion 400 with the internal gear 29. The internal gear 29 and the joint 28 function as an interlocking portion that interlocks with the operating portion 400 such that the state switches between the first state and the second state interlocking with the movement of the operating portion 400.

Specifically, the internal gear 29 and the joint 28 move part (pressed portion 21chb) of the leaf spring 21ch toward the held portion Wa interlocking with the movement of the operating portion 400 in a state where the catheter unit 100 is attached to the base unit 200. When the held portion 21chb moves, the coupling portion 21c is switched between the locked state and the unlocked state.

Alternatively, the internal gear 29 may be configured to be directly moved by the operating portion 400. In this case, the internal gear 29 has the function of an interlocking portion.

### Movement of Operating Portion

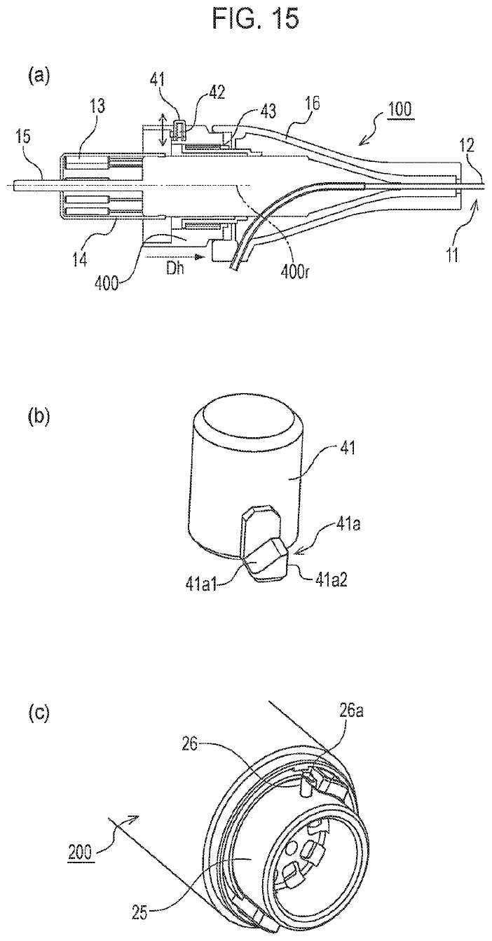

Movement of the operating portion 400 will be described with reference to FIGS. 15, 16, and 17.

In the present embodiment, the operating portion 400 is configured to be movable among the detachment position, the unlock position, and the fixed position in a state where the catheter unit 100 is attached to the base unit 200. The unlock position is located between the detachment position and the fixed position.

In the present embodiment, the operating portion 400 interlocks with the movement of the operating portion 400 between the unlock position and the fixed position, and the state is switched between the first state and the second state.

In the present embodiment, the operating portion 400 is movable between the detachment position and the fixed position by moving in a direction different from the attaching and detaching direction DE. The operating portion 400 moves in a direction that intersects with (preferably, a direction orthogonal to) the attaching and detaching direction DE to move between the detachment position and the fixed position. In the present embodiment, the operating portion 400 rotates around the rotation axis 400r extending in the attaching and detaching direction DE to move between the detachment position and the fixed position. Therefore, operability at the time when a user operates the operating portion 400 is good.

FIG. 15 is a view that illustrates the catheter unit 100 and the base unit 200. FIG. 15(a) is a sectional view of the catheter unit 100. FIG. 15(b) is a perspective view of a button 41. FIG. 15(c) is a perspective view of the base unit 200.

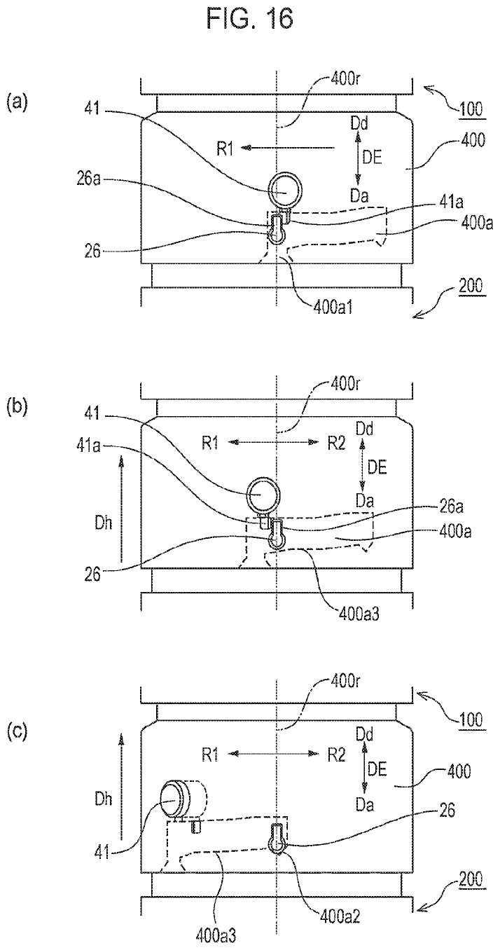

FIG. 16 is a view that illustrates the operation of the operating portion 400. FIG. 16(a) is a view that shows a state where the operating portion 400 is at the detachment position. FIG. 16(b) is a view that shows a state where the operating portion 400 is at the unlock position. FIG. 16(c) is a view that shows a state where the operating portion 400 is at the fixed position.

FIG. 17 is a sectional view that illustrates the operation of the operating portion 400. FIG. 17(a) is a sectional view that shows a state where the operating portion 400 is at the detachment position. FIG. 17(b) is a sectional view that shows a state where the operating portion 400 is at the unlock position. FIG. 17(c) is a sectional view that shows a state where the operating portion 400 is at the fixed position.

When the operating portion 400 is at the fixed position, the coupling portion 21c is in the locked state, and the held portion Wa of the drive wire W is fixed to the corresponding coupling portion 21c (see FIG. 14).

When the operating portion 400 is at the unlock position, the coupling portion 21c is in the unlocked state, and the locking of the held portion Wa of the drive wire W with the coupling portion 21c is unlocked (see FIG. 11). In this state, connection of the drive wire W with the wire drive portion 300 is interrupted. Therefore, at the time when the catheter 11 receives an external force, the bendable portion 12 can be freely bent without receiving resistance from the wire drive portion 300.

When the operating portion 400 is at the detachment position, the catheter unit 100 is allowed to be detached from the base unit 200. In a state where the operating portion 400 is at the detachment position, the catheter unit 100 is allowed to be attached to the base unit 200. When the operating portion 400 is at the detachment position, the coupling portion 21c is in the unlocked state, and the locking of the held portion Wa of the drive wire W with the coupling portion 21c is unlocked (see FIG. 10).

As shown in FIG. 15(a), the catheter unit 100 includes an operating portion urging spring 43 that urges the operating portion 400, the button 41 serving as a moving member, and a button spring 42 that urges the button 41.

In the present embodiment, the operating portion urging spring 43 is a compression spring. The operating portion 400 is urged in a direction Dh to approach the proximal end cover 16 by the operating portion urging spring 43.

In the present embodiment, the button 41 and the button spring 42 are included in the operating portion 400. When the operating portion 400 moves among the detachment position, the unlock position, and the fixed position, the button 41 and the button spring 42 move together with the operating portion 400.

The button 41 is configured to be movable with respect to the operating portion 400 in a direction that intersects with the direction of the rotation axis 400r of the operating portion 400. The button 41 is urged by the button spring 42 toward outside the catheter unit 100 (a direction to move away from the rotation axis 400r).

As will be described later, movement of the operating portion 400 from the unlock position to the detachment position is restricted by the button 41. When the button 41 is moved with respect to the operating portion 400, the operating portion 400 is allowed to move from the unlock position to the detachment position.

The button 41 has a button protrusion (restricted portion) 41a. The button protrusion 41a has a button slope 41a1 and a restricted surface 41a2.

The base unit 200 includes the base frame 25. The base frame 25 is provided with a lock shaft 26. The lock shaft 26 has a lock protrusion (restriction portion) 26a.

In the present embodiment, a plurality of (two in the present embodiment) the lock shafts 26 is provided. Each of the lock shafts 26 may have the lock protrusion 26a or one or some of the lock shafts 26 may have a lock protrusion 26a.

On the other hand, as shown in FIGS. 9, 16(a), 16(b), and 16(c), a lock groove 400a to be engaged with the lock shaft 26 is provided on the inner side of the operating portion 400. The lock groove 400a extends in a direction different from the attaching and detaching direction DE. In the present embodiment, the lock groove 400a extends in the rotation direction of the operating portion 400. The lock groove 400a may also be regarded as extending in a direction that intersects with (direction orthogonal to) the attaching and detaching direction DE.

When a plurality of the lock shafts 26 is provided, the lock groove 400a is provided for each of the plurality of lock shafts 26.

As shown in FIG. 16(a), when the catheter unit 100 is attached to the base unit 200, the lock shaft 26 engages with the lock groove 400a via the entrance 400a1 of the lock groove 400a.

At this time, the operating portion 400 is placed at the detachment position, and the coupling portion 21c is at the unlocked state (see FIG. 10). Therefore, this is a state where fixing of each of the first to ninth drive wires (W11 to W33) with a corresponding one of the first to ninth coupling portions (21c11 to 21c33) is unlocked. As shown in FIG. 17(a), the button protrusion 41a is opposite to the lock protrusion 26a.

When the operating portion 400 is rotated in a lock direction R1 in a state where the operating portion 400 is at the detachment position, the slope 41a1 of the button protrusion 41a contacts with a slope 26a1 of the lock protrusion 26a. The button 41 moves toward inside the operating portion 400 (a direction to approach the rotation axis 400r) against the urging force of the button spring 42. Then, the button protrusion 41a climbs over the lock protrusion 26a, and the operating portion 400 moves to the unlock position (see FIG. 17 (b)).

At this time, the coupling portion 21c is in the unlocked state (see FIG. 11). Therefore, this is a state where fixing of each of the first to ninth drive wires (W11 to W33) with a corresponding one of the first to ninth coupling portions (21c11 to 21c33) is unlocked.

In the present embodiment, even when the button 41 is not operated, the operating portion 400 is allowed to be moved from the detachment position to the unlock position. In other words, when the operating portion 400 is moved from the detachment position to the unlock position, a user does not need to operate the button 41.

When the operating portion 400 is rotated in the lock direction R1 in a state where the operating portion 400 is at the unlock position, the operating portion 400 moves to the fixed position. In a state where the operating portion 400 is at the fixed position, a positioning portion 400a2 of the lock groove 400a is placed at a position corresponding to the lock shaft 26. The operating portion 400 is urged in a direction Dh to approach the proximal end cover 16 by the operating portion urging spring 43. As a result, the positioning portion 400a2 is engaged with the lock shaft 26.

In the course in which the operating portion 400 moves from the unlock position to the fixed position, the held portion Wa of the drive wire W is fixed to the coupling portion 21c as described above.

In a state where the operating portion is placed at the fixed position, the coupling portion 21c is in the locked state (see FIG. 14). Therefore, each of the first to ninth drive wires (W11 to W33) is fixed to a corresponding one of the first to ninth coupling portions (21c11 to 21c33). In this state, driving force from the wire drive portion 300 can be transmitted to the bend drive portion 13. In other words, driving force from each of the first to ninth driving sources (M11 to M33) can be transmitted to a corresponding one of the first to ninth drive wires (W11 to W33) via a corresponding one of the first to ninth coupling portions (21c11 to 21c33).

When the operating portion 400 is at the unlock position, a wall 400a3 defining the lock groove 400a is placed on the upstream side of the lock shaft 26 in the detachment direction Dd of the catheter unit 100. When the operating portion 400 is at the fixed position, the positioning portion 400a2 is placed on the upstream side of the lock shaft 26 in the detachment direction Dd. As a result, when the operating portion 400 is at the unlock position or at the fixed position, detaching the catheter unit 100 from the base unit 200 is restricted. On the other hand, when the operating portion 400 is at the detachment position, the entrance 400a1 of the lock groove 400a is placed on the upstream side of the lock shaft 26 in the detachment direction Dd. As a result, detaching the catheter unit 100 from the base unit 200 is allowed.

When the operating portion 400 is rotated in an unlock direction R2 in a state where the operating portion 400 is at the fixed position, the operating portion 400 is placed at the unlock position. In the course in which the operating portion 400 moves from the fixed position to the unlock position, the held portion Wa of the drive wire W is unlocked from the coupling portion 21c as described above.

In a state where the operating portion 400 is placed at the unlock position, the restricted surface 41a2 of the button protrusion 41a contacts with the restriction surface 26a2 of the lock protrusion 26a (see FIG. 17(b)). In this state, rotating the operating portion 400 in the unlock direction R2 is restricted. Detaching the catheter unit 100 from the base unit 200 is restricted.

When a user pushes the button 41 toward inside the operating portion 400 in a state where the operating portion 400 is placed at the unlock position, the restricted surface 41a2 separates from the restriction surface 26a2, and the button protrusion 41a climbs over the lock protrusion 26a. As a result, the operating portion 400 is allowed to rotate in the unlock direction R2, and the operating portion 400 can be moved from the unlock position to the detachment position.

When the operating portion 400 is placed at the detachment position, the coupling portion 21c is in the unlocked state. Therefore, when the catheter unit 100 is detached from the base unit 200 or attached to the base unit 200, load (for example, resistance received by the coupling portion 21c) applied to the drive wire W is reduced. Therefore, a user is able to easily attach and detach the catheter unit 100.

When the operating portion 400 is placed at the unlock position, detaching the catheter unit 100 from the base unit 200 is restricted, and the coupling portion 21c is in the unlocked state. As described above, when the coupling portion 21c is in the unlocked state, connection of the drive wire W with the wire drive portion 300 is interrupted, and the bendable portion 12 can be freely bent without receiving resistance from the wire drive portion 300.

A user is able to stop the drive of the catheter 11 with the wire drive portion 300 by placing the operating portion 400 at the unlock position in a state where the catheter 11 is inserted in a target. In addition, since detaching the catheter unit 100 from the base unit 200 is restricted, a user is able to pull out the catheter 11 from inside the target while holding the base unit 200.

In the configuration of the present embodiment, when the button 41 is not operated, movement of the operating portion 400 from the unlock position to the detachment position is restricted. Therefore, when a user moves the operating portion 400 from the fixed position to the unlock position, erroneous movement of the operating portion 400 to the detachment position is reduced.

In the present embodiment, the number of the lock protrusions 26a and the number of the buttons 41 each are one. However, the medical device 1 may have a plurality of the lock protrusions 26a and a plurality of the buttons 41.

Detection Unit with Force Sensor

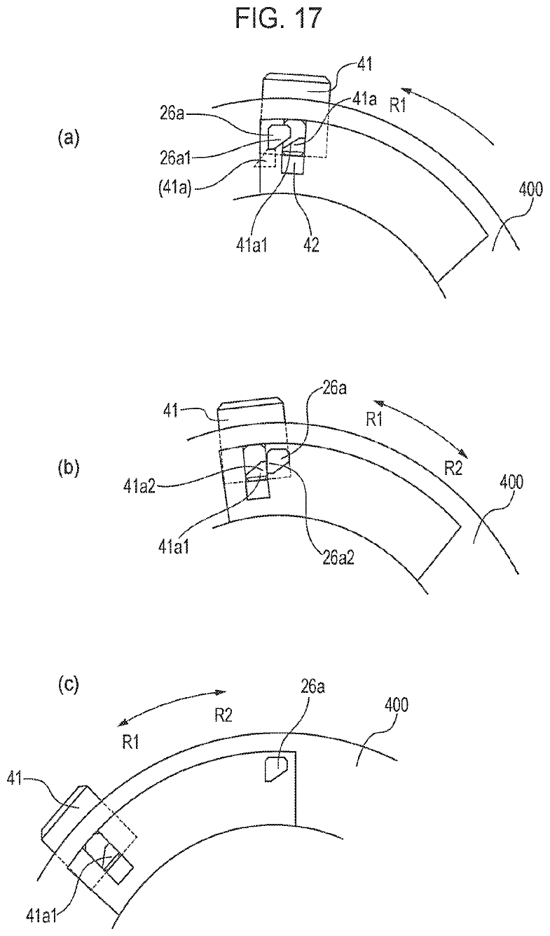

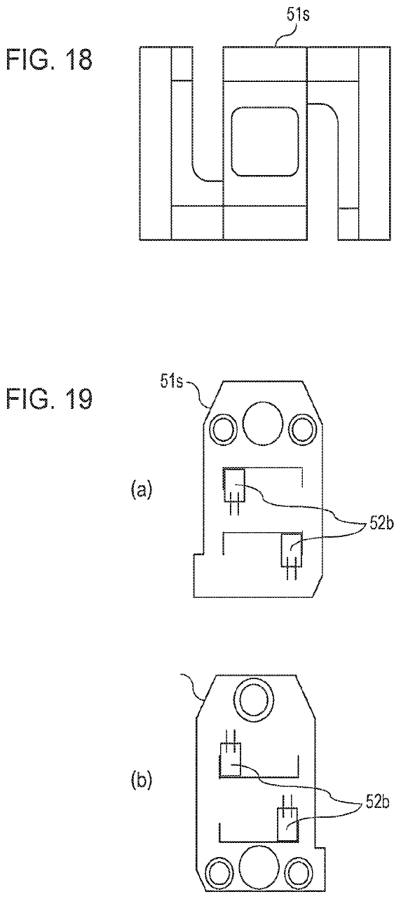

A detection unit will be described with reference to FIGS. 18, 19, and 20.

In the first embodiment, when a user uses the medical device, specifically, it is possible to detect that the coupling portion 21c is in the locked state and the drive wire W is coupled in a state where the catheter unit 100 is attached to the base unit 200 and in a state where the user has moved the operating portion 400 to the fixed position.

The detection unit includes a bridge circuit made up of a strain body that deforms according to an external force and strain gauges stuck to a deformable part that significantly receives a strain of the strain body. The detection unit includes a substrate with an amplifier that amplifies a signal corresponding to a strain and output from the bridge circuit.

FIG. 18 is side view that shows a strain body. The detection unit includes a plurality of strain bodies. In the first embodiment, the detection unit includes the first strain body 51s11, the second strain body 51s12, the third strain body 51s13, the fourth strain body 51s21, the fifth strain body 51s22, the sixth strain body 51s23, the seventh strain body 51s31, the eighth strain body 51s32, and the ninth strain body 51s33.

Of the first to ninth strain bodies (51s11 to 51s33), a selected one may be referred to as a strain body 51s. In the first embodiment, each of the first to ninth strain bodies (51s11 to 51s33) has the same shape.

Each of the plurality of strain bodies 51s is connected to a corresponding one of the plurality of driving sources M via a corresponding one of the tractor support shafts 21cs, with the result that the driving force of each of the plurality of driving sources M is given as an external force. In the first embodiment, each of the plurality of strain bodies 51s is connected between the driving source M and the coupling portion 21c. Specifically, the first strain body 51s11 is connected to the first driving source M11. The second strain body 51s12 is connected to the second driving source M12. The third strain body 51s13 is connected to the third driving source M13. The fourth strain body 51s21 is connected to the fourth driving source M21. The fifth strain body 51s22 is connected to the fifth driving source M22. The sixth strain body 51s23 is connected to the sixth driving source M23. The seventh strain body 51s31 is connected to the seventh driving source M31. The eighth strain body 51s32 is connected to the eighth driving source M32. The ninth strain body 51s33 is connected to the ninth driving source M33.

As described above, the strain body 51s is connected to the coupling portion 21c. The bend drive portion 13 receives the driving force of the wire drive portion 300 via the coupling device 21 and the strain body 51s and bends the bendable portion 12. Specifically, the first strain body 51s11 is connected to the first coupling portion 21c11. The second strain body 51s12 is connected to the second coupling portion 21c12. The third strain body 51s13 is connected to the third coupling portion 21c13. The fourth strain body 51s21 is connected to the fourth coupling portion 21c21. The fifth strain body 51s22 is connected to the fifth coupling portion 21c22. The sixth strain body 51s23 is connected to the sixth coupling portion 21c23. The seventh strain body 51s31 is connected to the seventh coupling portion 21c31. The eighth strain body 51s32 is connected to the eighth coupling portion 21c32. The ninth strain body 51s33 is connected to the ninth coupling portion 21c33.

FIG. 19(a) is a front view that shows the strain body 51s to which strain gauges are stuck. FIG. 19(b) is a back view that shows the strain body 51s to which strain gauges are stuck.

The detection unit includes a bridge circuit made up of strain gauges. In the first embodiment, the detection unit includes a first bridge circuit 52b11, a second bridge circuit 52b12, a third bridge circuit 52b13, a fourth bridge circuit 52b21, a fifth bridge circuit 52b22, a sixth bridge circuit 52b23, a seventh bridge circuit 52b31, an eighth bridge circuit 52b32, and a ninth bridge circuit 52b33.

Of the first to ninth bridge circuits (52b11 to 52b33), a selected one may be referred to as a bridge circuit 52b. In the first embodiment, each of the first to ninth bridge circuits (52b11 to 52b33) has the same configuration.

Each of the plurality of bridge circuits 52b is stuck to a corresponding one of the plurality of strain bodies 51s. Specifically, the first bridge circuit 52b11 is stuck to the first strain body 51s11. The second bridge circuit 52b12 is stuck to the second strain body 51s12. The third bridge circuit 52b13 is stuck to the third strain body 51s13. The fourth bridge circuit 52b21 is stuck to the fourth strain body 51s21. The fifth bridge circuit 52b22 is stuck to the fifth strain body 51s22. The sixth bridge circuit 52b23 is stuck to the sixth strain body 51s23. The seventh bridge circuit 52b31 is stuck to the seventh strain body 51s31. The eighth bridge circuit 52b32 is stuck to the eighth strain body 51s32. The ninth bridge circuit 52b33 is stuck to the ninth strain body 51s33.

A strain gauge may be stuck by adhesive, vapor deposition, or other techniques. A strain gauge is stuck to a position where deformation of the strain body 51s is easily detected. A strain gauge similarly deforms with deformation of the strain body 51s. At this time, the electric resistivity of the strain gauge changes with the amount of deformation, and the bridge circuit 52b outputs the change in resistance value.

In the first embodiment, a four-gauge method is used in the configuration of the bridge circuit made up of strain gauges. Alternatively, a one-gauge method, a two-gauge method, or other methods may be used.

In the first embodiment, a bridge circuit made up of strain gauges is used for a force sensor. Alternatively, a force sensor of another type, such as an electrostatic capacitance type and a piezoelectric type, may be used.

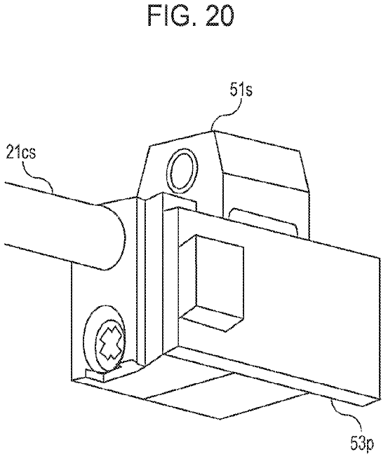

As described above, strain gauges that make up a force sensor are stuck to the strain body 51s. Thus, the bridge circuit is formed. To handle its output, the detection unit includes a plurality of substrates. FIG. 20 is a perspective view that shows a substrate stuck to the strain body. In the first embodiment, the detection unit includes the first substrate 53p11, the second substrate 53p12, the third substrate 53p13, the fourth substrate 53p21, the fifth substrate 53p22, the sixth substrate 53p23, the seventh substrate 53p31, the eighth substrate 53p32, and the ninth substrate 53p33.

Of the first to ninth substrates (53p11 to 53p33), a selected one may be referred to as a substrate 53p. In the first embodiment, each of the first to ninth substrates (53p11 to 53p33) has the same configuration.

Each of the plurality of substrates 53p is connected to conductors extended out from the strain gauges attached to a corresponding one of the strain bodies 51s. It is possible to detect whether coupling is made by obtaining an output signal according to a strain from the bridge circuit made up of the strain gauges in accordance with deformation of the strain body 51s. Specifically, the first substrate 53p11 detects a strain of the first strain body 51s11. The second substrate 53p12 detects a strain of the second strain body 51s12. The third substrate 53p13 detects a strain of the third strain body 51s13. The fourth substrate 53p21 detects a strain of the fourth strain body 51s21. The fifth substrate 53p22 detects a strain of the fifth strain body 51s22. The sixth substrate 53p23 detects a strain of the sixth strain body 51s23. The seventh substrate 53p31 detects a strain of the seventh strain body 51s31. The eighth substrate 53p32 detects a strain of the eighth strain body 51s32. The ninth substrate 53p33 detects a strain of the ninth strain body 51s33.

A strain gauge is stuck to a position where deformation of the strain body is easily detected. A strain gauge similarly deforms with deformation of the strain body. At this time, the electric resistivity of the strain gauge changes with the amount of deformation, and the bridge circuit outputs the change in resistance value. When the substrate obtains the output value, the output value is amplified by an amplifier, and an external force is detected in accordance with the amplified value.

In the first embodiment, the detection unit is connected between the tractor support shaft 21cs and the coupling base 21cb to be configured to detect an external force. The strain body 51s is formed so as to be deformed by an external force in a Dc direction. The strain body 51s is connected by the tractor support shaft 21s and receives the driving force of the driving source M via the tractor 21ct. Strain gauges are stuck to the front side and back side of the strain body 51s to make up a bridge circuit, and conductors extended out from the strain gauges are connected to the substrate 53p.

In the first embodiment, the strain body 51s deforms upon receiving driving force resulting from the drive of the driving source M, and the electric resistivity of each of the strain gauges changes. A minute electrical signal generated as a result of the change in electric resistivity is given to the amplifier through the conductor connected to each strain gauge, with the result that an amplified signal is obtained.

### Detection Steps

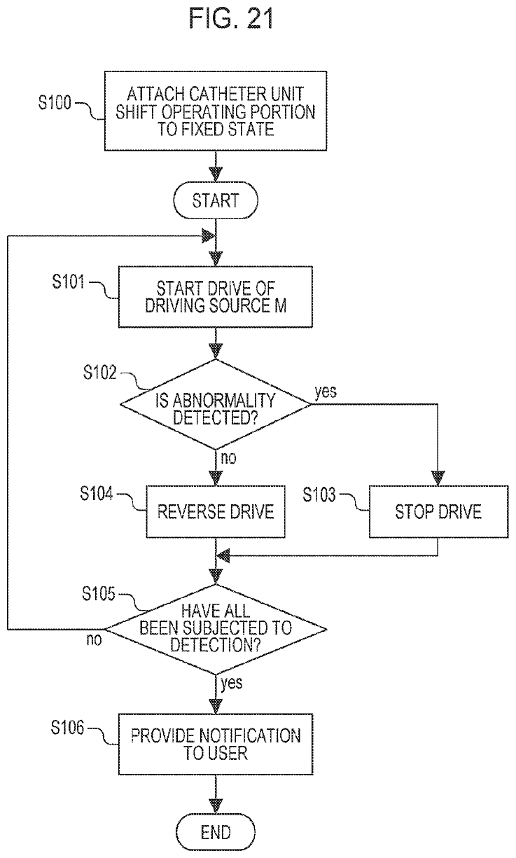

FIG. 21 is a flowchart of a series of steps in which the detection unit according to the first embodiment performs detection. Detection steps related to the medical device and the detection unit used in the medical device according to the first embodiment will be described with reference to FIG. 21.

In the detection steps, it is assumed that, as shown in step S100, a user attaches the catheter unit 100 to the base unit 200 and moves the operating portion 400 to the fixed position.

Initially, one driving source M (normally, the first driving source M11) is driven in a set direction (step S101). It is detected whether the coupling portion 21c is coupled or abnormal while the driving source M is being driven (step S102). When it is detected as being abnormal, the drive is immediately terminated (step S103). When it is detected as being not abnormal, the driving source M is reversed to return to a state before being driven (step S104).

The series of steps is performed on all the driving sources M and the coupling portions 21c, and it is also detected which one of the driving source M and the coupling portion 21c is abnormal (step S105). After that, notification about which driving source M and which coupling portion 21c are normal or abnormal is provided to the user (step S106).

The detection steps related to the medical device and the detection unit used in the medical device according to the first embodiment have been described above with reference to FIG. 21. As described above, in the first embodiment, it is detected whether the coupling portion 21c is normally coupled. When it is detected as being abnormal, the drive is immediately terminated and stopped as shown in step S103. Therefore, a user is able to safely perform work thereafter for the user him or herself and a patient. Since it is detected which driving source M and which coupling portion 21c are abnormal, it is easy to check for abnormal locations.

FIG. 21 illustrates that notification is provided to a user after all the detection ends for the sake of description. Alternatively, actual steps do not need to be performed in this way. For example, when the drive of one driving source M is terminated, notification related to the coupling portion 21c connected to the driving source M may be provided.

Next, among the detection steps shown in FIG. 21, an abnormality detection unit using the detection unit will be described in detail.

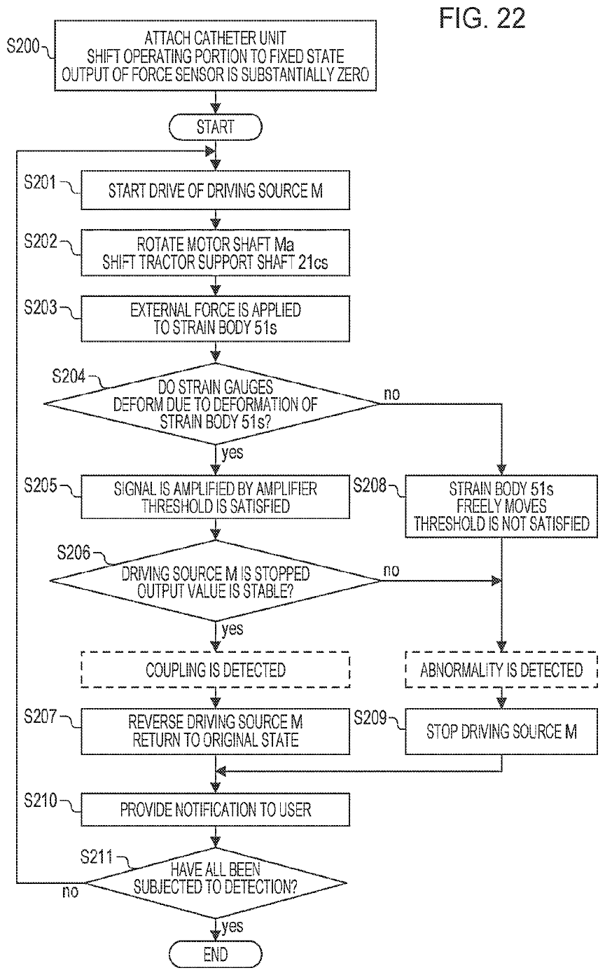

Steps of the abnormality detection unit based on the strain body 51s to which the bridge circuit 52b made up of strain gauges are stuck, which is an example of the abnormality detection unit, will be described with reference to FIG. 22. Beforehand, the detection unit has a determination threshold as to whether coupling is made. The determination threshold is determined according to a value that the bridge circuit 52b outputs when the coupling portion 21c is definitely coupled and the driving source M is driven. Specifically, the determination threshold is determined as a threshold by which it is possible to discriminate a difference between the output of the bridge circuit when coupling is made and the output of the bridge circuit when coupling is not made.

Normally, in a state where the catheter unit 100 is attached by a user, the bendable portion is in a straight state. This means that no external force is applied and the output value of the bridge circuit 52b that is the force sensor is substantially zero (step S200). In abnormality detection, detection is performed for each coupling portion 21c and each driving source M.

Initially, driving the driving source M in a set direction for a predetermined time is started (step S201). The predetermined time is a time until the output value reaches the determination threshold, or until the tractor 21ct contacts with the end portion of a screw, or determined by a configuration. As described with reference to FIG. 18, when the driving source M is driven, the motor shaft Ma rotates. Accordingly, the tractor 21ct moves in the Dc direction. With movement of the tractor 21ct, the tractor support shaft 21cs moves in the Dc direction (step S202).

As a result, the external force in the Dc direction is applied to the strain body 51s (step S203). At this time, it is detected whether coupling is made in accordance with whether a value that satisfies the determination threshold is output from the bridge circuit 52b as a result of deformation of the strain body 51s and deformation of the strain gauges (step S204).

When coupling is made, the strain body 51s receives an external force from the driving source M and a force as a reaction from the drive wire W via the coupling portion 21c to deform. Through the deformation, as described with reference to FIG. 19, the strain gauges stuck to the strain body 51s deform. Thus, the output of the bridge circuit 52b changes. The output value of the bridge circuit 52b is connected to the substrate 53p through the conductors extended out from the strain gauges. Since the output value of the bridge circuit 52b is a minute signal, the output value is amplified to a detectable value by using an amplifier. While driving is continued, deformation of the strain body 51s increases, and the output value of the bridge circuit 52b satisfies the determination threshold (step S205). When it is determined that the determination threshold is satisfied through comparison, the driving source M is temporarily stopped. When the output value satisfies the determination threshold during the stop and stability is ensured, it is detected that coupling is made (step S206). Then, the driving source M is reversed and is reversed until the output value of the bridge circuit 52b becomes zero, that is, the bendable portion is returned to a straight state before detection (step S207).

When coupling is not made, the strain body 51s is supported only by the tractor support shaft 21cs. In other words, the strain body 51s freely moves as in the case of the tractor support shaft 21cs. At this time, since the strain body 51s almost does not deform, the output of the bridge circuit 52b is substantially zero. For this reason, even when the driving source M is driven for the predetermined time, the determination threshold is not satisfied, so it is detected that coupling is not made (step S208). In this case, the driving source M is immediately stopped (step S209). After detection, notification about whether it is normal or abnormal is provided to the user (step S210). After that, for the subsequent coupling portion 21c and the subsequent driving source M as well, detection is performed repeatedly in the above steps. Thus, detection of all the coupling portions 21c and all the driving sources M is performed.

Since a user is able to check for coupling of each coupling portion 21c in a state where the catheter unit 100 is attached to the base unit 200, there is an advantageous effect of preventing a malfunction during coupling and a redo during work is obtained.

In the above description, the catheter unit 100 is assumed to be attached to the base unit 200 just before operation, and is in a straight state with no external force applied. Alternatively, detection may be performed in a state where the catheter unit 100 is curved. During operation or the like, the catheter unit 100 may be once taken out from inside a patient, and detection may be performed again at the time when the catheter unit 100 is inserted again.

### Second Embodiment

Detection Unit with Current Sensors

Next, a detection unit with current sensors will be described.

The detection unit according to a second embodiment is also basically similar to the first embodiment. In the following description, different points between the embodiments will be mainly described, and the description of similar portions will not be repeated.

The detection unit according to the second embodiment includes a plurality of current sensors capable of detecting coupling of the catheter unit 100 with the base unit 200 by measuring the drive current of each driving source M. In the second embodiment, the detection unit includes the first current sensor 54s11, the second current sensor 54s12, the third current sensor 54s13, the fourth current sensor 54s21, the fifth current sensor 54s22, the sixth current sensor 54s23, the seventh current sensor 54s31, the eighth current sensor 54s32, and the ninth current sensor 54s33.

Of the first to ninth current sensors (54s11 to 54s33), a selected one may be referred to as a current sensor 54s. In the second embodiment, each of the first to ninth current sensors (54s11 to 54s33) has the same configuration.

Each of the plurality of current sensors 54s is connected to a corresponding one of the plurality of driving sources M to measure a corresponding one of drive currents. Specifically, the first current sensor 54s11 is connected to the first driving source M11. The second current sensor 54s12 is connected to the second driving source M12. The third current sensor 54s13 is connected to the third driving source M13. The fourth current sensor 54s21 is connected to the fourth driving source M21. The fifth current sensor 54s22 is connected to the fifth driving source M22. The sixth current sensor 54s23 is connected to the sixth driving source M23. The seventh current sensor 54s31 is connected to the seventh driving source M31. The eighth current sensor 54s32 is connected to the eighth driving source M32. The ninth current sensor 54s33 is connected to the ninth driving source M33.

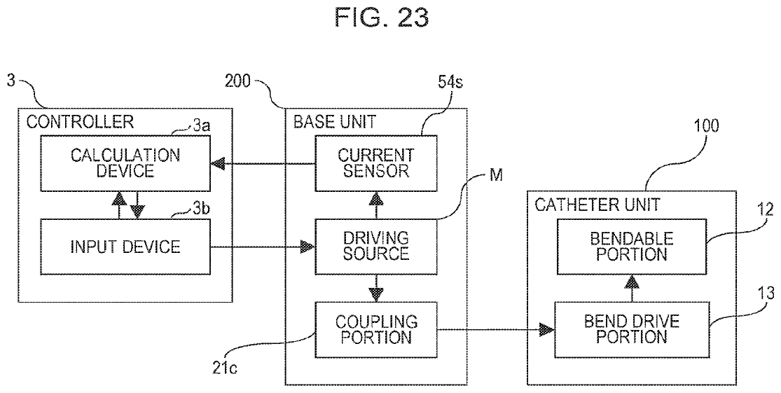

FIG. 23 is a block diagram of coupling detection with the current sensors. The driving source M drives the bendable portion 12 to bend upon receiving a command from the input device 3b of the controller 3. To properly bend the bendable portion 12, the bend drive portion 13 and the coupling portion 21c need to be coupled to each other. For this purpose, the drive current of the driving source M is monitored by the current sensor 54s to detect whether coupling is normal.

Specifically, coupling detection is performed by measuring a current from the input device 3b to the driving source M, inputting the measured current value to the calculation device 3a, and comparing the input with the determination threshold. After that, the input device 3b provides an appropriate command to the driving source M.

Other than coupling detection, when, for example, the output value of drive current is abnormally large or no current is passing, these situations may be detected because they are abnormal.

A current sensor may perform detection in accordance with a voltage drop of a known resistor, may use magnetism, or may adopt other methods.

Next, an abnormality detection unit using the detection unit with current sensors will be described in detail.

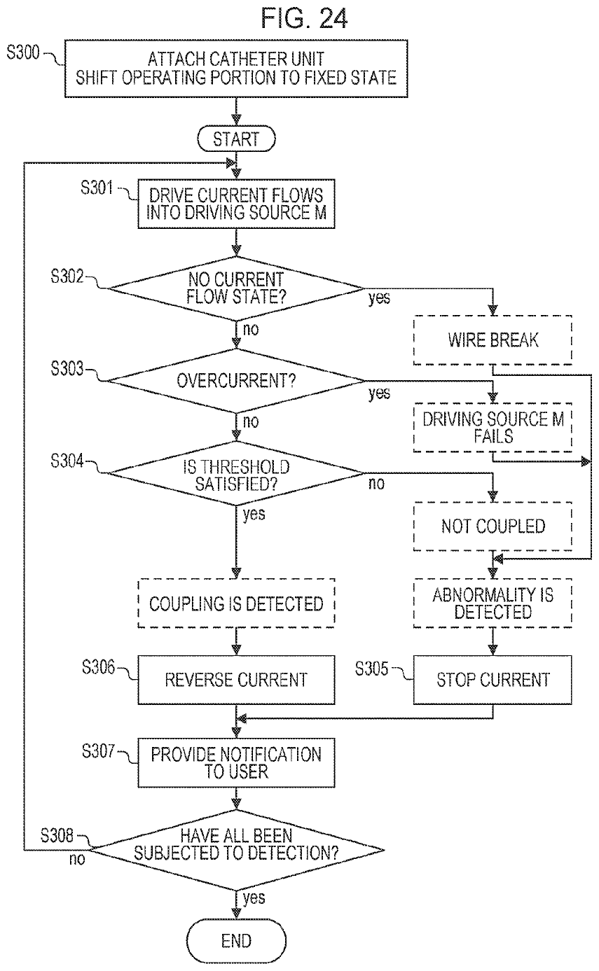

Steps of the abnormality detection unit based on measurement of drive current of each driving source M with the current sensors 54s, which are examples of the abnormality detection unit, will be described with reference to FIG. 24. Beforehand, the detection unit has a determination threshold as to whether coupling is made. The determination threshold is determined according to a value of current needed to drive the driving source M at the time when the coupling portion 21c is definitely coupled. Specifically, when coupling is not made, a load connected to the driving source M is small, so the drive current is small. On the other hand, when coupling is made, the catheter unit 100 is coupled to the driving source M, so a load is large. Therefore, the drive current for driving the driving source M further increases as compared to when coupling is not made. The determination threshold is determined as a threshold by which it is possible to discriminate a difference between these values.

Normally, in a state where a user attaches the catheter unit 100 and moves the operating portion 400 to the fixed state (step S300), abnormality detection is performed for each coupling portion 21c and each driving source M.

Initially, driving the driving source M in a set direction for a predetermined time is started, that is, a drive current is input to the driving source M (step S301). The predetermined time is a time until the output value reaches the determination threshold, or until the tractor 21ct contacts with the end portion of a screw, or determined by a configuration.

Subsequently, detection of coupling is performed by monitoring the value of the current sensor 54s. Detection as to whether no current passes (step S302), detection as to whether it is overcurrent (step S303), and detection as to whether the threshold is satisfied (step S304) are performed. When it is detected that no current is passing, overcurrent is passing, or coupling is not made, it is suspected that, other than the fact that coupling is not made, there is a break, a short circuit, a failure of the driving source M, or the like, so current is immediately stopped (step S305). When it is detected that coupling is made, current is reversed to return to an original state (step S306).

After detection, notification about whether it is normal or abnormal is provided to the user (step S307). After that, for the subsequent coupling portion 21c and the subsequent driving source M as well, detection is performed repeatedly in the above steps. Thus, detection of all the coupling portions 21c and all the driving sources M is performed (step S308).

Since a user is able to check for coupling of each coupling portion 21c in a state where the catheter unit 100 is attached to the base unit 200, there is an advantageous effect of preventing a malfunction during coupling and a redo during work is obtained. In addition, a failure of the driving source M and a break can be checked, so it is possible to immediately terminate work.

### Third Embodiment

Detection Unit with Pass/Fail Determination Unit on Catheter Next, a pass/fail determination unit on a catheter will be described.

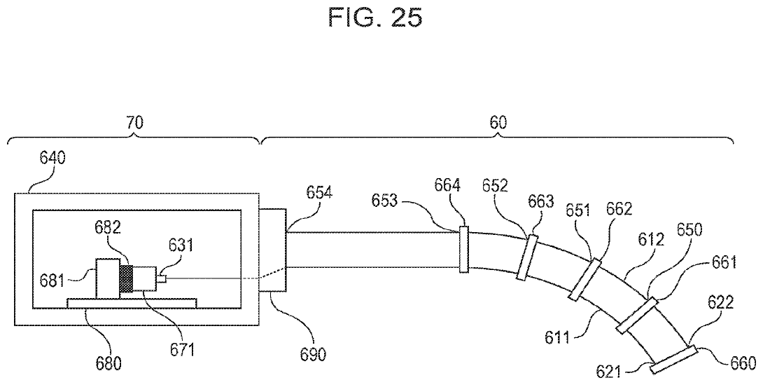

FIG. 25 is a view that shows a schematic configuration example of a continuum robot according to a third embodiment of the present invention. The continuum robot is made up of a bendable portion 60 that bends a distal end portion and a drive portion 700 that drives the continuum robot.

A wire 611 and a wire 612 are respectively connected to a fixed portion 621 and a fixed portion 622 at a distal end 660 of the bendable portion 60. The bendable portion 60 is configured to have wire guides 661 to 664 that are members for guiding the wire 611 and the wire 612. The wire guides are not limited to a method of discretely disposing a plurality of members. Alternatively, a continuum member, such as an accordion shape and a mesh shape, may be used. The wire guides 661 to 664 are fixed to the wire 612 at fixed portions 650 to 654.

The drive portion 70 is made up of a robot base portion 640, a wire holding mechanism 671 that supports a wire holding pipe 631, and an actuator 680. The wire holding mechanism 671 is connected to a movable portion 681 of the actuator 680 via a wire tension detecting portion 682, and is movable back and forth.

The bendable portion 60 is detachably attachable to the drive portion 70 via the connecting portion 690 and replaceable, and the proximal end of the wire 611 of the bendable portion 60 is connected to the wire holding pipe 631 in the robot base portion 640. In addition, a connection position of the wire 611 with the wire holding mechanism 671 may be shifted. At that time, the position of the wire 611 is converted so as to be aligned with the position of the wire holding mechanism 671 at the connecting portion 690 and connected. The position of the bendable portion 60 is controlled by pushing or pulling the wire holding pipe 631 with the actuator 680.

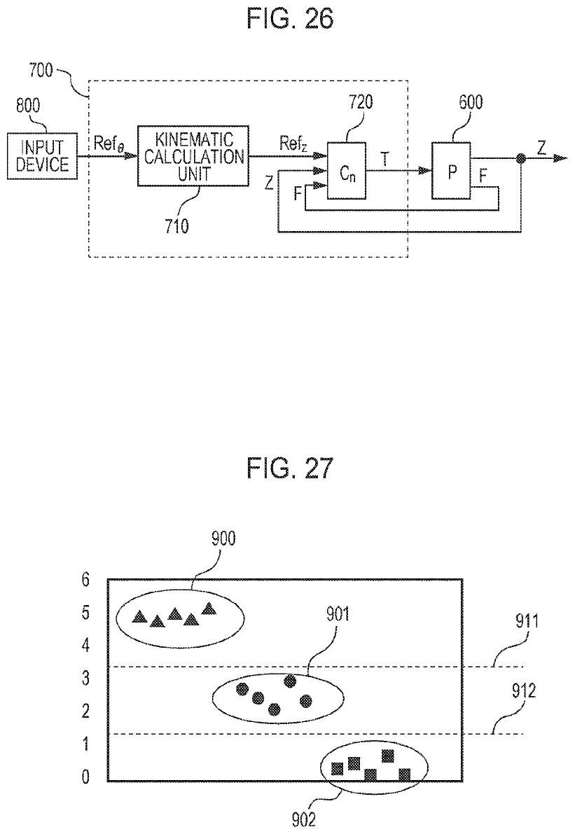

FIG. 26 is a schematic configuration example of a control system of the continuum robot according to the embodiment of the present invention. The control system of the continuum robot is configured to include a continuum robot 600 that is a controlled target, a controller 700 for the continuum robot, and an input device 800. The controller 700 for the continuum robot is configured to include a kinematic calculation unit 710, and a control unit 720. The control unit 720 feeds back position information Z of the wire and the tension F of the wire, acquired from the continuum robot 600, performs position control calculation or force control calculation, and outputs a torque command T to the continuum robot 600. A bend angle command Ref_{θ} of the bendable portion is output from the input device 800, and the kinematic calculation unit converts the bend angle command Ref_{θ} to a position command Ref_{Z} of the movable portion 681 of the actuator 680 and outputs the position command Ref_{Z} to the control unit 720. Thus, positioning the bendable portion 60 of the continuum robot 600 to an intended position and control for a set force are implemented.

When the bendable portion 60 corresponding to the catheter described in the first embodiment and the second embodiment and the drive portion 70 corresponding to the base unit are attached to each other, connection with the wire 611 and the wire holding pipe 631 is made. At this time, when there is a connection mistake, the bendable portion is not moved according to the bend angle Ref_{θ} provided as a command from the input device 800. When the slidability of the wire 611 on the wire guides 661 to 664 is lower than a prescribed value due to individual differences in the bendable portion 60, the bendable portion 60 is not controlled with a force provided as a command during force control. Next, a method of detecting a wire connection mistake and poor slidability of wire guides when the bendable portion 60 is attached will be escribed.

A torque command T with a selected frequency is output from the control unit 720 to the continuum robot 600, and the average value of the amplitude of the wire position Z at this time is recorded. In the present embodiment, a torque command with a frequency of 1 Hz is output, and the average value of the amplitude of the wire position Z in 10 seconds is divided by the input torque. Thus, a displacement amplitude per unit torque is obtained. Hereinafter, the amount of displacement (amplitude per unit torque) is described as the slidability of the continuum robot. FIG. 27 shows the slidability of the continuum robot according to the present invention. The ordinate axis represents the slidability of the continuum robot, and shows the slidability under a condition 900 in which the wire 611 and the wire holding pipe 631 are not connected, a condition 901 in which the wire 611 and the wire holding pipe 631 are normally connected, and a condition 902 in which a defective bendable portion 60 with a low wire guide slidability is connected. In the condition 900 in which the wire 611 and the wire holding pipe 631 are not connected, the slidability is the highest. By connecting the wire 611 and the wire holding pipe 631, the slidability of the wire 611 on the wire guides 661 to 664 is added, so the slidability decreases (condition 901). By connecting the defective bendable portion 60 with a low wire guide slidability, the slidability further decreases (condition 902).

When the slidability is higher than a wire connection determination threshold 911, it is determined that the wire 611 and the wire holding pipe 631 are not connected. When the slidability is lower than a wire guide poor slidability determination threshold 912, it is determined that the wire guide slidability of the bendable portion 20 is poor. When the slidability is between the wire connection determination threshold 911 and the wire guide poor slidability determination threshold 912, the wire 611 and the wire holding pipe 631 are normally connected, and it is determined that the wire guide slidability of the bendable portion 20 is good.

When the bendable portion 20 is detachably attachable and replaceable, the slidability is measured as described above. A determination as to a connection mistake between the wire and the wire holding pipe and pass/fail of the bendable portion 20 is performed in accordance with the measurement result. Notification about the result is provided to an operator of the continuum robot.

As a method of investigating the slidability of the continuum robot, a wire tension detected by the wire tension detecting portion 682 may be used. A torque command with a frequency of 1 Hz is output, the average value of the amplitude of wire tension in 10 seconds is divided by the input torque. The slidability may be evaluated by obtaining a tension amplitude per unit torque.

### REFERENCE SIGNS LIST

100: catheter unit
200: base unit
21: coupling device
21c: coupling portion
400: operating portion
41: button
51s: strain body
52b: bridge circuit (force sensor)
53p: substrate
54s: current sensor
W: drive wire
Wa: held portion
600: continuum robot
700: controller
800: input device

1. A device comprising:
a base including a driving source and a coupling portion;
a bendable body detachably attached to the base, the bendable body including a bendable portion configured to bend and a drive wire allowed to be coupled to the coupling portion and configured to bend the bendable portion;
a force sensor provided in the base and that is connected to the coupling portion, the force sensor being connected to the drive wire while the drive wire is coupled to the coupling portion; and
a detector configured to detect a state of coupling of the coupling portion with the drive wire,
wherein while the base is coupled with the coupling portion, the detector detects the state of coupling using an output value of the force sensor while the drive wire is driven by the driving source.

2. The device according to claim 1, wherein
a plurality of the driving sources, a plurality of the coupling portions, and a plurality of the drive wires are provided, and
wherein the detector is provided to detect coupling of each of the plurality of coupling portions with a corresponding one of the plurality of drive wires.

3. The device according to claim 1, wherein
the bendable body includes a plurality of the bendable portions.

4. The device according to claim 1, wherein
the detector determines whether an output of the force sensor satisfies a threshold.

5. The device according to claim 4, wherein
in a case where the threshold is not satisfied, driving of the driving source is stopped, and notification that an abnormality is detected is provided to a user.

6. The device according to claim 4, wherein
in a case where the threshold is satisfied, the bendable portion is returned to a straight state before detection by reversing drive of the driving source.

7. The device according to claim 1, further comprising:
a position detector for the drive wire; and
wherein the detector determines a state of coupling of the coupling portion with the drive wire based on an amount of displacement of a position detected by the position detector, the displacement being caused by driving the drive wire to be pushed and pulled with a predetermined force.

8. The device according to claim 1, wherein
the detector determines a slidability of the drive wire in the bendable body in accordance with an amount of displacement of a position detected by the position detector, the displacement being caused by driving the drive wire to be pushed and pulled by the driving source with a predetermined force.

9. The device according to claim 1, further comprising:
a current sensor configured to measure a drive current of the driving source; and
wherein, the detector determines a state of coupling of the coupling portion with the drive wire based on an output of the current sensor during a driving of the driving source.

10. An actuator comprising:
a driving source;
a coupling portion,
wherein a bendable body is detachably attached to the actuator via the coupling portion, the bendable body including a bendable portion configured to bend and a drive wire allowed to be coupled to the coupling portion and configured to bend the bendable portion;
a force sensor that is connected to the coupling portion, the force sensor being connected to the drive wire while the drive wire is coupled to the coupling portion; and a detector configured to detect a state of coupling of the coupling portion with the drive wire,
wherein while the base is coupled with the coupling portion, the detector detects the state of coupling using the output value of the force sensor while the drive wire is driven by the driving source.

11. The actuator according to claim 10, wherein
the detector determines whether an output of the force sensor satisfies a threshold.

12. The actuator according to claim 11, wherein
in a case where the threshold is satisfied, the bendable portion is returned to a straight state before detection by reversing drive of the driving source.

13. The actuator according to claim 10, further comprising:
a position detector for the drive wire; and
wherein the detector determines a state of coupling of the coupling portion with the drive wire based on an amount of displacement of a position detected by the position detector, the displacement being caused by driving the drive wire to be pushed and pulled with a predetermined force.

14. The actuator according to claim 10, wherein
the detector determines a slidability of the drive wire in the bendable body in accordance with an amount of displacement of a position detected by the position detector, the displacement being caused by driving the drive wire to be pushed and pulled by the driving source with a predetermined force.

15. The actuator according to claim 10, further comprising:
a current sensor configured to measure a drive current of the driving source; and
wherein, the detector determines a state of coupling of the coupling portion with the drive wire based on an output of the current sensor during a driving of the driving source.
